(19) United States
(12) Patent Application Publication
(10) Pub. No.: US 2010/0083964 A1
(43) Pub. Date: Apr. 8, 2010

(54) DISPENSER DOSE COUNTER MECHANISM

(75) Inventors: Scott Brown, Princeton, NJ (US); Aleksandr Zuyev, Morganville, NJ (US); Stephen Miggels, Wyckoff, NJ (US); Henry J. Mack, JR., Phillipsburg, NJ (US); Mikhail Gotliboym, Scotch Plains, NJ (US); In-Young Jang, Ridgefield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/520,069

(22) PCT Filed: Dec. 21, 2007
(86) PCT No.: PCT/US2007/026214

§ 371 Date: Dec. 8, 2009

Related U.S. Application Data
(60) Provisional application No. 60/871,681, filed on Dec. 22, 2006.

(51) Int. Cl. A61M 15/00; A61M 15/08

(52) U.S. Cl. 128/203.15

(57) ABSTRACT

An apparatus for counting doses of a dispenser is provided which includes a housing for releasably attaching to the dispenser; and, a dose counter operatively connected to the housing or the dispenser. The dose counter displays indicia relating to doses of the dispenser. The dose counter may be manually adjusted or configured to be automatically adjusted to permit counting of the number of times the housing is separated from the dispenser. The number of separations will indicate the number of doses administered by the dispenser. Advantageously, with the subject invention, a dose counter is provided which counts doses separately from the actuation of the dispenser. As will be appreciated by those skilled in the art, the invention may apply to various dispensers, but is particularly well-suited for use with inhalers.

### BACKGROUND OF THE INVENTION

This invention relates to dose-counting mechanisms, more particularly, to dose-counting mechanisms for inhalers.

Inhalers are commonplace and used extensively throughout society, including for regimented drug delivery, delivery of drug on an as-needed basis, and emergency drug delivery. Inhalers can include oral inhalers and inhalers, which may be spray atomizers, dry powder inhalers (DPI's), or metered dose inhalers (MDI's). An inhaler may or may not use a propellant. For example, a MDI delivers with a propellant a measured amount of medication as a mist which a patient inhales. A DPI makes available a dry plug or plugs of medicament which is inhaled without a propellant; under force of inhalation, the dry plug(s) break(s) up in the DPI, thereby delivering a fine dry powder to the user. Also, an inhaler can be formed as a spray atomizer, which causes liquid to break up and form a mist without the use of a propellant. Typically, MDI's and DPI's are used for oral inhalation, while MDI's and spray atomizers are used for nasal inhalation. People with asthma, chronic obstructive pulmonary disease (COPD), chronic bronchitis, and emphysema are typical users of inhalers.

Inhalers are formed with opaque bodies or drug canisters which do not permit visual determination of the number of available doses for delivery. Failure to properly count the number of administered doses may lead to an unexpected depletion. This is particularly risky where an individual suffers from an acute or life-threatening condition and requires the administration of a drug by an inhaler. As a result of the potential dangers associated with the inability to visually monitor the available number of doses, the United States Food and Drug Administration has recommended that MDI's include integrated dose-counting mechanisms to count the number of administered doses to permit a user to evaluate the number of remaining doses. See, U.S. Department of Evaluation and Research (CDER), Clinical Medical, Guidance for Industry, Integration of Dose-Counting Mechanisms Into MDI Drug Products (March, 2003).

Integrated dose counting mechanisms have been developed in the prior art for use with MDI's, such as that disclosed in U.S. Pat. No. 5,482,030 to Klein; U.S. Pat. No. 5,718,355 to Garby et al.; U.S. Pat. No. 5,988,496 to Bruna; U.S. Pat. No. 6,082,358 to Scarrott et al.; U.S. Pat. No. 6,752,153 to Eckert; PCT Published Application No. WO 98/56446; and, PCT Published Application No. WO 2005/113044. All of the cited documents disclose an integrated dose counting mechanism which relies on the activation of the MDI for counting of a dose. In particular, the dose counting mechanism relies on the movement of the drug canister during activation of the MDI to actuate the dose counting mechanism.

### SUMMARY OF THE INVENTION

In various embodiments of the subject invention, a dose counter is provided which counts doses separately from the actuation of the dispenser. As will be appreciated by those skilled in the art, the subject invention may be applied to various dispensers, but is particularly well-suited for use with inhalers.

In one aspect of the subject invention, an apparatus for counting doses of a dispenser is provided which includes a housing for releasably attaching to the dispenser; and, a manually adjustable dose counter operatively connectable to the dispenser. The dose counter displays indicia relating to doses of the dispenser.

In a further aspect of the subject invention, an apparatus for counting doses of a dispenser is provided, with the apparatus including a housing for releasably attaching to the dispenser; an adjustable dose counter operatively connectable to the dispenser, the dose counter displaying indicia relating to doses of the dispenser; and, means for automatically causing adjustment of the dose counter upon the housing being attached to, or detached from, the dispenser.

In yet a further aspect of the subject invention, an apparatus for counting doses of a dispenser is provided. With the dispenser having a dispense opening through which the doses are administered, the apparatus includes a housing for releasably attaching to the dispenser, the housing being configured to cover at least a portion of the dispense opening when attached to the dispenser; and, a dose counter operatively connectable to the dispenser, the dose counter displaying indicia relating to doses of the dispenser.

These and other features of the invention will be better understood through a study of the following detailed description and accompanying drawings.

### BRIEF DESCRIPTION OF THE DRAWINGS

FIGS. 1-10 depict a manually adjustable dose counter of the subject invention;

FIGS. 11-20 depict an automatically adjustable dose counter of the subject invention;

FIGS. 21-32 depict an automatically adjustable dose counter of the subject invention;

FIGS. 33-37 depict an electronic dose counter of the subject invention;

FIG. 38 depicts the invention in use with a nasal inhaler;

FIG. 39 depicts a tether usable with the subject invention; and

FIGS. 40-46 depict preferred embodiments of the subject invention.

### DETAILED DESCRIPTION OF THE INVENTION

The subject invention provides a dose counter for use with various dispensers. As will be recognized by those skilled in the art, the subject invention is particularly well-suited for use with inhalers, including, but not limited to, MDI's, DPI's and spray atomizers. For illustrative purposes, the drawings and following description refer to an inhaler, particularly a MDI. It is to be understood that various configurations of dispensers, particularly inhalers, may be utilized in conjunction with the invention. This includes not only other types of inhalers, e.g., nasal inhalers, but other configurations of inhalers. By way of non-limiting example, the subject invention may be utilized in conjunction with an oral inhaler sold under the trademark “PROVENTIL” by Schering-Plough Corporation or may be used in conjunction with a nasal inhaler sold under the trademark “NASONEX” by Schering-Plough Corporation.

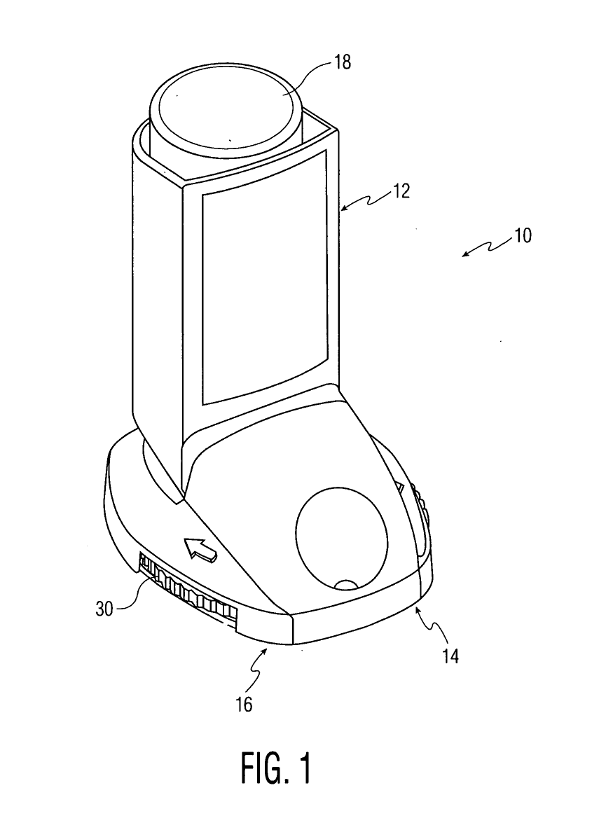

With reference to FIG. 1, a kit 10 is shown which includes a dispenser 12 and a housing 14. The housing 14 is formed to be releasably attached to the dispenser 12 in any known matter. Preferably, the housing 14 is formed with a generally flat resting surface 16 sufficiently sized to provide a stable resting surface for the kit 10 when assembled. Advantageously, the resting surface 16 permits the dispenser 12 to be kept in an upright position between uses. As such, the internal valving of the dispenser 12 can be better maintained in a wet condition and primed than with the dispenser 12 being kept on its side or upside-down. In addition, the housing 14 can be formed to hold the dispenser 12 in a forward-leaning state when attached. With this configuration, any unadministered dose remnants can be gravitationally urged to flow towards the housing 14. It is believed that the internal valving of the dispenser 12 can be kept in a cleaner state in this manner.

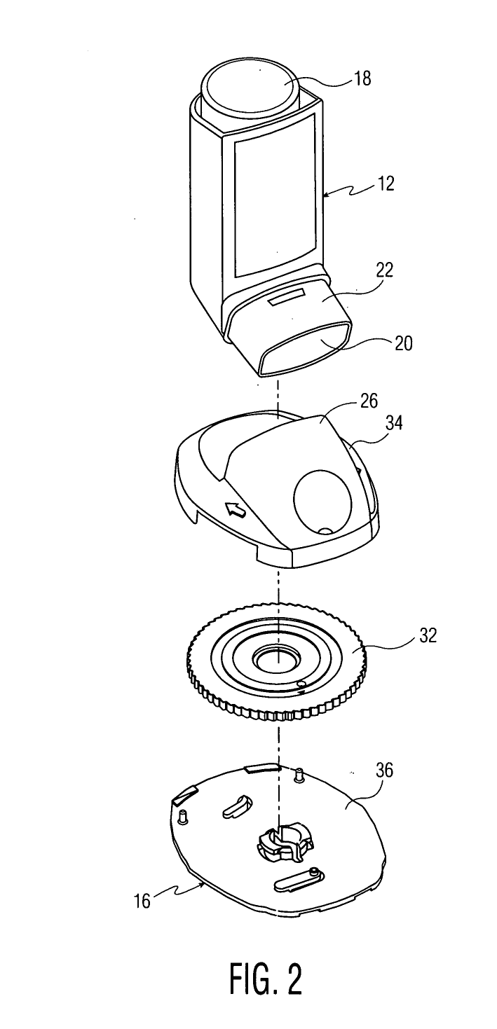

As indicated above, the dispenser 12 may be of various configurations. With reference to FIGS. 1 and 2, the dispenser 12 will include a drug reservoir 18 formed to accommodate a drug to be delivered. Suitable medicaments include an anticholinergic, a corticosteroid, a long acting beta agonist, short acting beta agonist, a phosphodiesterase IV inhibitor and combinations of two or more thereof. Suitable medicaments may be useful for in the prevention or treatment of a respiratory, inflammatory or obstructive airway disease. Suitable anticholinergics include (R)-3-[2-hydroxy-2,2-(dithien-2-yl)acetoxy]-1-1[2-(phenyl)ethyl]-1-azoniabicyclo[2.2.2]octane, Glycopyrrolate, Ipratropium Bromide, Oxitropium Bromide, Atropine Methyl Nitrate, Atropine Sulfate, Ipratropium, Belladonna Extract, Scopolamine, Scopolamine Methobromide, Methscopolamine, Homatropine Methobromide, Hyoscyamine, Isopriopramide, Orphenadrine, Benzalkonium Chloride, Tiotropium Bromide, GSK202405, an individual isomer of any of the above or a pharmaceutically acceptable salt or hydrate of any of the above, or a combination of two or more of the above. Suitable corticosteroids includes Mometasone Furoate; Beclomethasone Dipropionate; Budesonide; Fluticasone; Dexamethasone; Flunisolide; Triamcinolone; (22R)-6.alpha., 9.alpha.-difluoro-11.beta., 21-dihydroxy-16.alpha., 17. alpha.-propylmethylenedioxy-4-pregnen-3,20-dione, Tipredane, GSK685698, GSK799943 or a pharmaceutically acceptable salt or hydrate of any of the above, or a combination of two or more of the above. Suitable long acting beta agonist include carmoterol, indacaterol, TA-2005, salmeterol, formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, pirbuterol acetate or a pharmaceutically acceptable salt or hydrate of any of the above, or a combination of two or more of the above. Suitable phosphodiesterase IV inhibitors include Cilomilast, Roflumilast, Tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above. Albuterol and mometasone furoate monohydrate are commonly used in inhalers.

A dispense opening 20 is formed in the dispenser 12 through which doses of drug accommodated in the drug reservoir 18 are administered. With the dispenser 12 being an oral inhaler, the dispenser 12 will include a mouthpiece 22, in which the dispense opening 20 is formed. As is known in the art, the drug reservoir 18 may be formed to be replaceable or not replaceable, depending on the re-usability of the dispenser 12. As a MDI, the drug reservoir 18 may be a pressurized cartridge or canister which permits a dose of the drug to be delivered in a mist. Any mechanism for delivering the drug from the drug reservoir 18 and through the dispense opening 20 may be utilized with the subject invention.

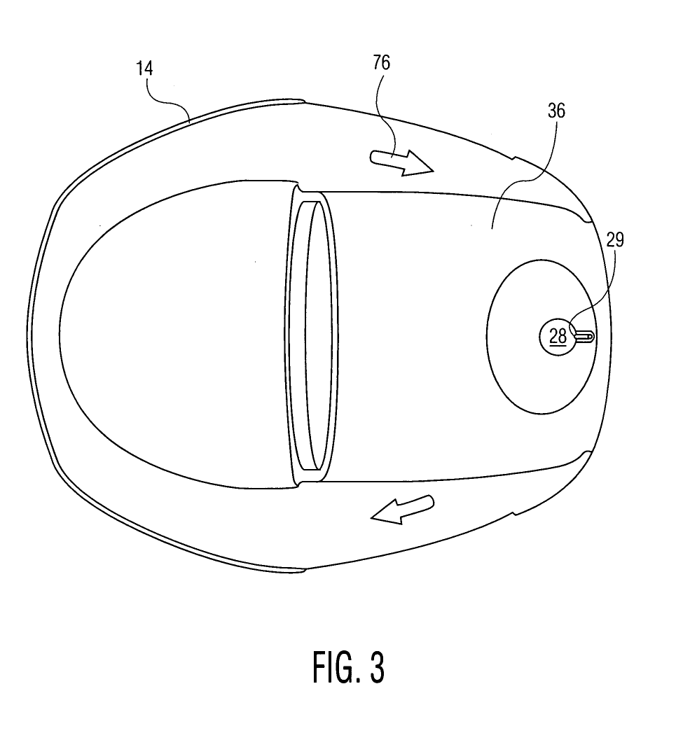

With reference to FIG. 3, it is preferred that the housing 14 be formed with an aperture 24 sized to receive a portion of the dispenser 12, more preferably to receive a portion of the dispenser 12 with the dispense opening 20. It is further preferred that a canopy 26 be formed in the housing 14 formed to cover at least a portion of, more preferably to cover wholly, the dispense opening 20 with the dispenser 12 extending through the aperture 24. The canopy 26 permits the kit 10 to be stored with the dispense opening 20 being shielded from the open atmosphere. The canopy 26 may be band shell-shaped or take other shapes.

As indicated above, it is preferred that the housing 14 be releasably attachable to the dispenser 12. The releasable attachment may be defined by a frictional connection or mechanical connection (e.g., snap fit) between the aperture 24 and the dispenser 12. In addition, or alternatively, the aperture 24 may be formed to interferingly engage (e.g., interference fit) the dispenser 12. Cooperating members may also be formed on the dispenser 12 and the housing 14 which can be locked together, such as cooperating threads; a bayonet lock; etc. Any form of releasable engagement (e.g., mechanical interaction; adhesive; chemical attraction; magnetic attraction) may be utilized which generates sufficient holding force to prevent inadvertent detachment of the dispenser 12 from the housing 14, but permits separation thereof for a user.

The housing 14 may be also formed with a window 28 through which the number of doses can be displayed as described below. A pointer 29 may extend into the window 28 to point out a particular indicium.

A dose counter 30 (FIG. 1) is operatively connected to the housing 14 or the dispenser 12 to allow a user to count doses administered by the dispenser 12. Various embodiments of the dose counter 30 may be utilized. For example, an analog or digital display may be used to indicate the number of doses. In addition, the dose counter 30 may be manually adjusted or automatically adjusted upon the dispenser 12 being attached to, or detached from, the housing 14. The following description provides possible configurations for the dose counter 30. As will be readily appreciated by those skilled in the art, other configurations consistent with the disclosure herein, may also be utilized.

With reference to FIGS. 1 and 2, a first variation of the dose counter 30 is shown which is manually adjustable. The dose counter 30 includes a counter 32 accommodated inside of the housing 14. To simplify assembly, the housing 14 may be formed of two portions, an upper cap 34 and a lower base 36, which are attached in any known manner. The resting surface 16 may be formed on the external surface of the lower base 36.

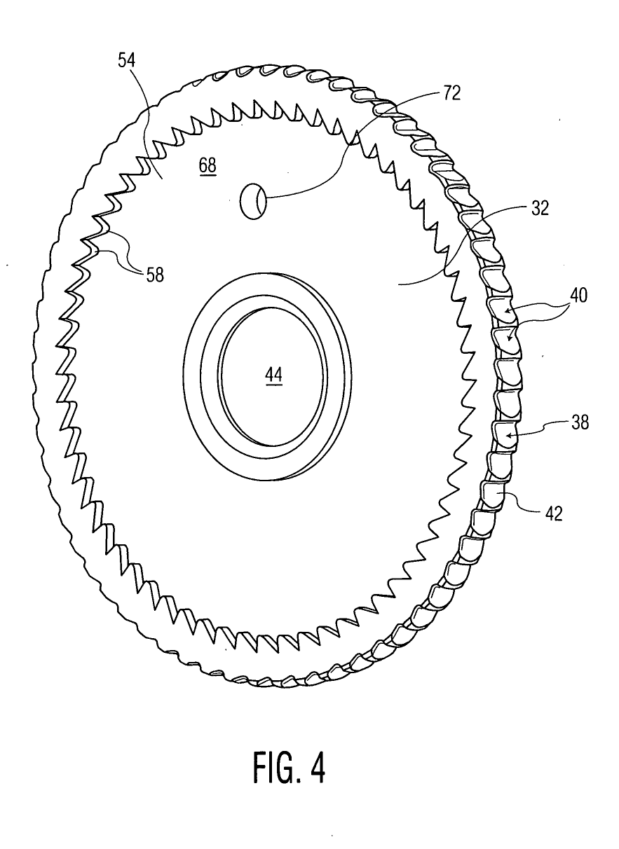

As shown in FIG. 4, the counter 32 is formed with an outer edge 38 which is preferably formed with a series of teeth 40. Preferably, the teeth 40 are formed to define outwardly-facing engaging surfaces 42 which all extend with the same radial orientation about the counter 32 (i.e., all the engaging surfaces 42 extend in either a clockwise or counterclockwise direction). In this manner, the teeth 40 are formed to more easily permit rotation of the counter 32 in one radial direction, but not in a reverse direction. The teeth 40 are intended to provide enhanced frictional engagement of the counter 32 for a user. Other configurations to enhance frictional engagement are possible on the outer edge 38 including providing a textured or knurled surface.

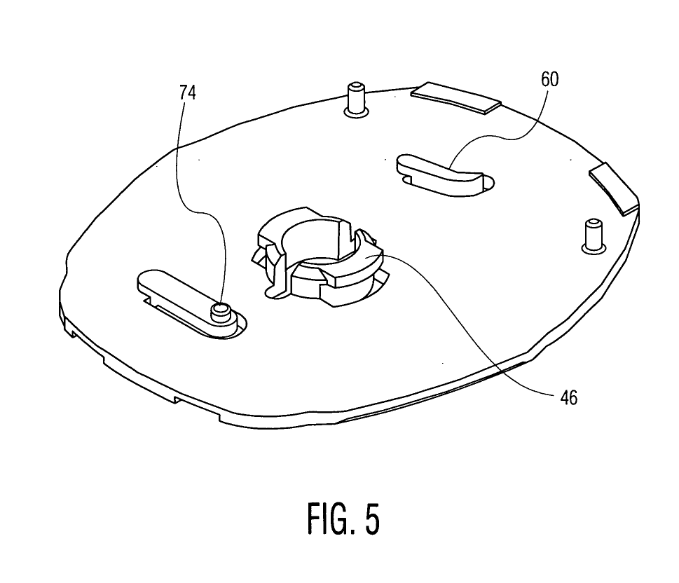

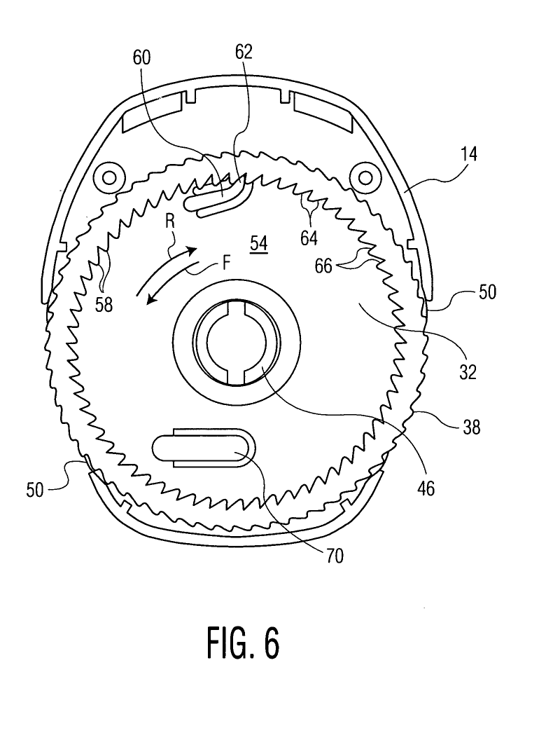

The counter 32 is rotatably coupled to the housing 14. To achieve this coupling, the counter 32 may be formed with an inner opening 44 that is engaged by latches 46 extending from the lower base 36 (FIGS. 5-7). The latches 46 may be inwardly deflectable to permit mounting of the counter 32 thereabout. Lips 48 may be formed on the latches 46 to prevent separation of the counter 32 from the latches 46 once mounted. With reference to FIG. 6, the housing 14 is formed with at least one, preferably two, side openings 50. The counter 32 is sized and positioned on the lower base 36 to have portions of the outer edge 38 be accessible through the side openings 50. With the housing 14 being assembled, and the counter 32 being located therein, the side openings 50 permit access to the counter 32. With access, the counter 32 may be manually adjusted so as to be rotated about the latches 46.

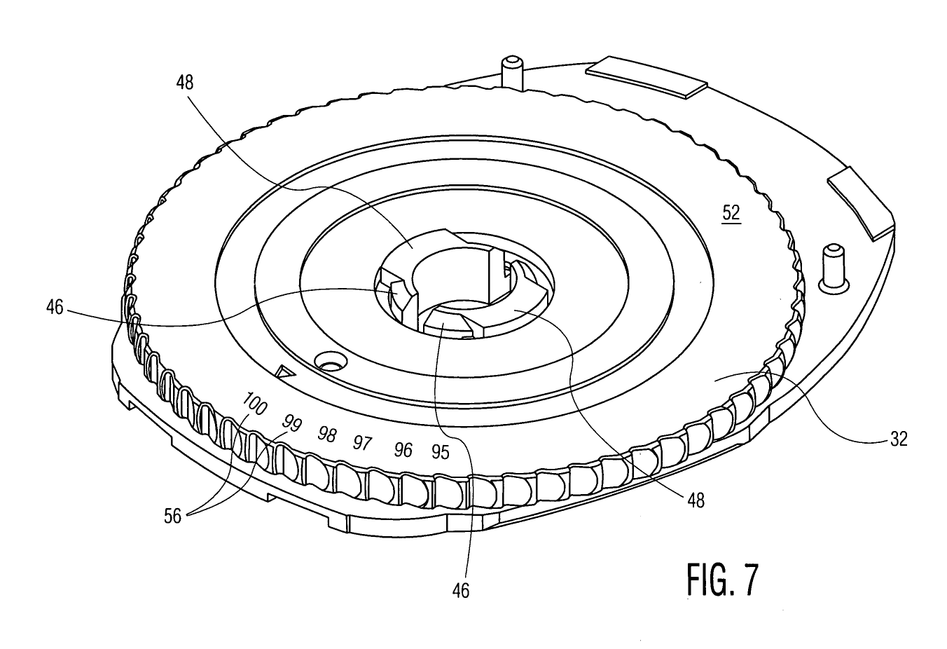

The counter 32 is formed with an upper face 52 and a lower face 54. The upper face 52 is oriented away from the lower base 36 and toward the upper cap 34 in an assembled state. As shown in FIG. 7, indicia 56 may be disposed on the upper face 52 sized and positioned to be viewable through the window 28 formed in the housing 14. Preferably, the window 28 and the indicia 56 are configured to permit viewing of only a single of the indicia 56 at one time to avoid confusion. The indicia 56 may be of any alpha-numeric characters. Preferably, the indicia 56 is a numeric series which permits iteratively to count down the number of remaining doses in the dispenser 12. Thus, the indicia 56 would have as a first number in a series the total number of doses in the dispenser (e.g., 100 doses) with a series of numbers decreasing by one to zero. With each dose administration, the counter 32 is rotatably adjusted to indicate one less dose remains in the dispenser 12. The indicia 56 may be adapted in other manners: to count the number of administered doses (i.e., to count up rather than down); to count daily doses; to indicate days of the week; to count general levels of remaining doses (e.g., counting by 10's); etc. The indicia 56 may also include graphics and colored fields or backgrounds. For example, the indicia 56 may be consecutive bands of color indicating the general state of the number of available doses: a green band may indicate a relatively high number of doses; a yellow band may indicate an intermediate number of doses; and, a red band may indicate a low number of doses. Optionally, colors may be applied as background to alpha-numeric characters to permit the reading of available number of doses and to simultaneously provide an indication of the general state of the dispenser 12.

The indicia 56 may be arrayed in any fashion. With the counter 32 having a wheel shape, the indicia 56 may be arranged in a ring. The indicia 56 may also be arranged in parallel, e.g., where the counter 32 is shiftable, to permit a higher number of the indicia 56 to be provided (e.g., one ring of the indicia indicating 1-50 doses may be provided adjacent to a ring of indicia indicating 51-100 doses; with a shift of the counter 32, the corresponding ring may be utilized). In addition, more than one of the counters 32 may be provided which can be shifted under a gearing mechanism. Furthermore, a plurality of the counters 32 may be arranged and adjusted in parallel to indicate different numbers which collectively correspond to a dose (e.g., three counters each indicating a one's value, a ten's value and a hundred's value). With multiple arrays and/or a plurality of the counters 32, a high number of the indicia 56 may be provided.

Preferably, the dose counter 30 includes a mechanism which only permits one-way rotation of the counter 32 to minimize the possibility of the counter 32 rotating in the wrong direction. More preferably, the dose counter 30 also includes a locking mechanism for releasably locking the dose counter 30 to selectively prevent adjustment thereof. As such, inadvertent rotation may be prevented. By way of non-limiting example, a mechanism for permitting one-way rotation and releasable locking is depicted. The counter 32 on the lower face 54 is provided with a ring of inwardly facing ratchet teeth 58, as shown in FIGS. 4 and 6. The ratchet teeth 58 are preferably saw-tooth shaped and oriented in the same radial direction. The ratchet teeth 58 are radially oriented to prevent rotation of the counter 30 in a direction opposite to the direction of rotation of the counter 32 needed to properly adjust the indicia 56. An indexer 60 extends from the lower base 36 which has an end 62 shaped to nest between two of the ratchet teeth 58. The indexer 60 is radially biased towards the ratchet teeth 58 so as to have a state at rest with the end 62 nesting between the ratchet teeth 58. The nested state of the end 62 inhibits rotation of the counter 32 and provides a locking effect. With the slanted configuration of the ratchet teeth 58, the ratchet teeth 58 have sloped surfaces 64 and generally straight back surfaces 66. Each of the back surfaces 66 and the end 62 of the indexer 60 are configured to meet in general flat face-to-face engagement which prevents the indexer 60 from ascending any of the back surfaces 66 thereby preventing the ratchet teeth 58 from passing the end 62. The interengagement of the back surfaces 66 and the end 62 prevents rotation of the counter 32 in a rearward direction (represented by arrow R in FIG. 6). The end 62 and each of the sloped surfaces 64 are formed to permit the end 62 to ascend the sloped surfaces 64 under force of rotation of the counter 32 in the proper direction needed to adjust the rotation 56 with the end 62 eventually bypassing the sloped surface 64. Once past the sloped surface 64, and under inherent force of bias, the end 62 nests between an adjacent pair of the back surface 66 and the sloped surface 64. With this arrangement, backward rotation of the counter 32 is prohibited while a locking effect is provided which can be overcome by force of rotation of the counter 32 in a forward rotation (represented by arrow F in FIG. 6).

To minimize the overall volume of the dose counter 30, it is preferred that the ratchet teeth 58 be provided in proximity to the outer edge 38 with a void 68 (FIG. 4) being defined therewithin. The indexer 60 may extend from the lower base 36 and into the void 68.

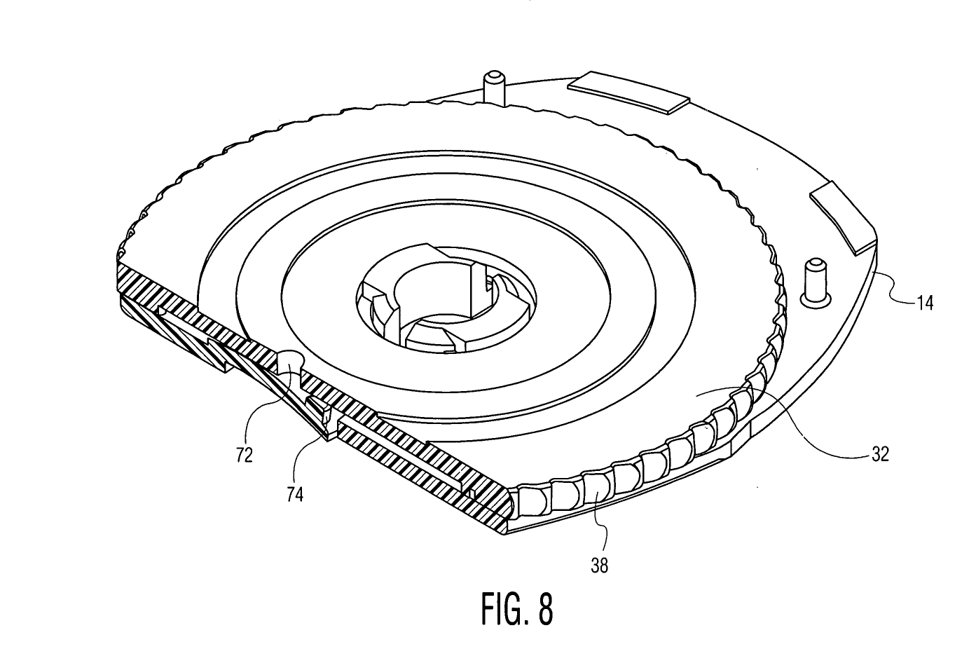

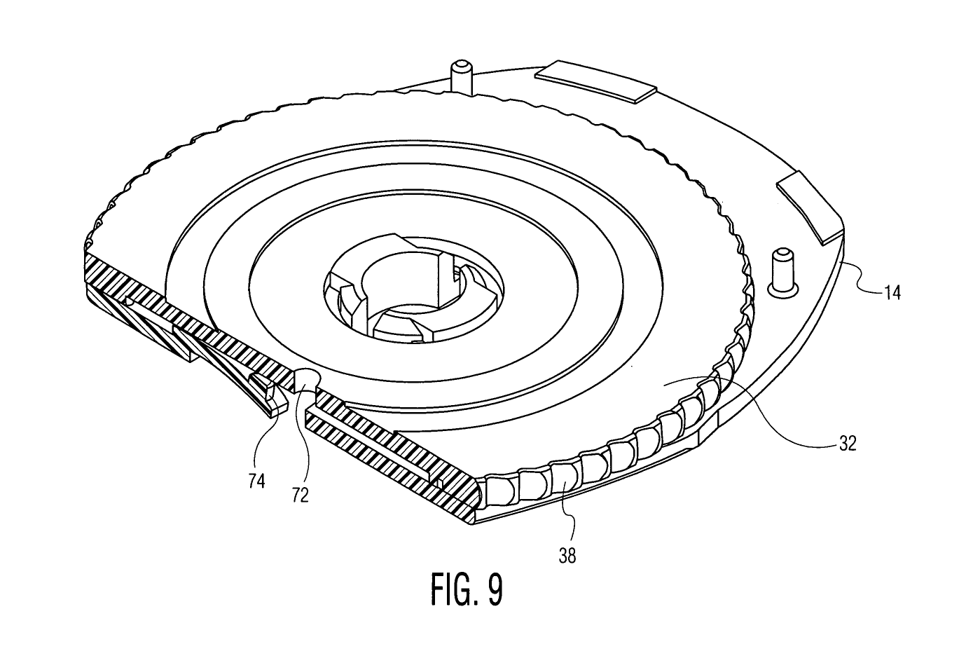

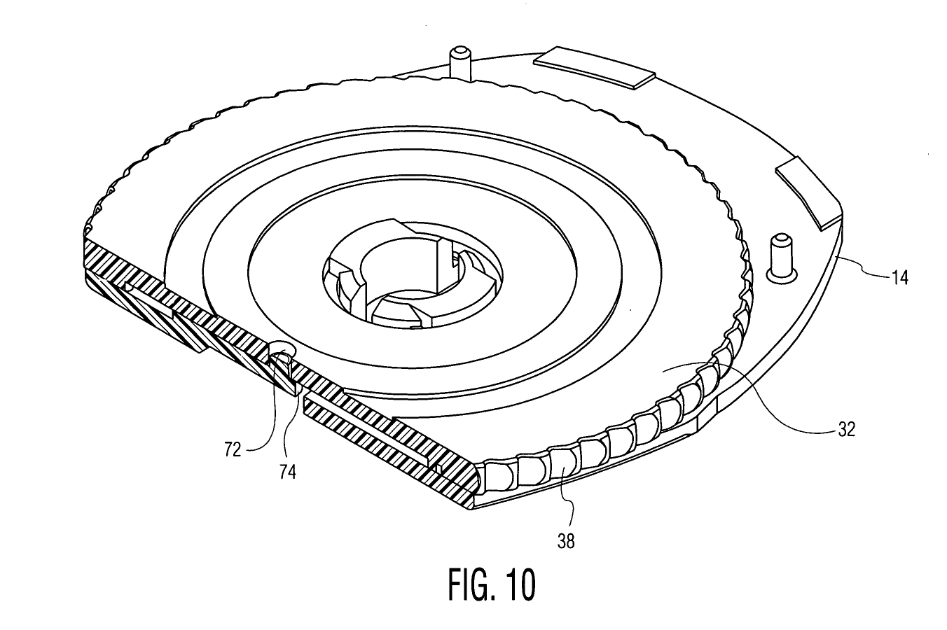

The dose counter 30 may be further provided with a lock 70 which is configured to be engaged upon full depletion of the doses in the dispenser 12. The lock 70 completely prohibits rotation of the counter 32 in either direction. With reference to FIG. 4, the lock 70 may include a locking aperture 72 in the counter 32 positioned and configured to be engaged by locking detent 74 (FIG. 5) extending from the housing 14, preferably extending from the lower base 36. With reference to FIGS. 8-10, the locking aperture 72 is spaced from the locking detent 74 at a start position of the dose counter 30 (i.e., a position coinciding with the beginning of dose counting). The locking aperture 72 is radially spaced from the locking detent 74 in the same radial direction needed to adjust the counter 32 during operation thereof. The locking detent 74 is biased upwardly towards the counter 32. With the locking aperture 72 being out of alignment with the locking detent 74, the locking detent 74 presses against the counter 32. During use, the locking aperture 72 rotates away from the locking detent 74. As the number of doses in the dispenser 12 is depleted, the locking aperture 72 approaches the locking detent 74, as shown in FIG. 9. Upon dose depletion, the locking aperture 72 comes into alignment with the locking detent 74, as shown in FIG. 10. In alignment, the locking detent 74 is biased into insertion into the locking aperture 72. The interengagement of the detent 74 in the locking aperture 72 prevents rotation of the counter 32 in either direction.

The housing 14 may be provided as a single-use item which is discarded with the dispenser 12 upon dose depletion. Alternatively, the housing 14 may be re-usable to be used with a plurality of the dispensers 12. For re-use, the lock 70 may not be provided or be formed to be re-settable, such as to allow disengagement of the locking detent 74 from the locking aperture 72. To assist a user, directional arrows 76 (FIG. 2) may be provided on the housing 14 to indicate proper direction or rotation necessary to adjust the dose counter 30.

During use, the housing 14 is separated from the dispenser 12 to permit drug administration. A user adjusts the dose counter 30 one increment relative to the window 28 before or after using the dispenser 12. Preferably, the ratchet teeth 58 and the indexer 60 are configured to limit movement of the dose counter 30 to one increment intervals.

Other configurations of the dose counter 30 are usable with the subject invention which are operatively connected to the housing 14. The dose counter 30 can be formed to be automatically adjusted upon the housing 14 being attached to, or detached from, the dispenser 12. In a second variation of the dose counter 30, the dose counter 30 is formed to be automatically adjusted upon attachment of the dispenser 12 to the housing 14.

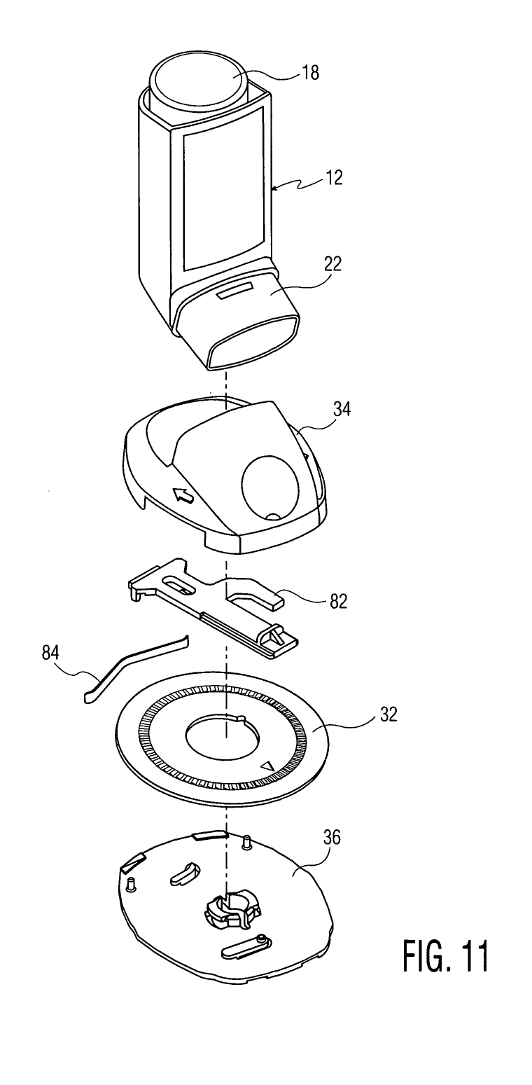

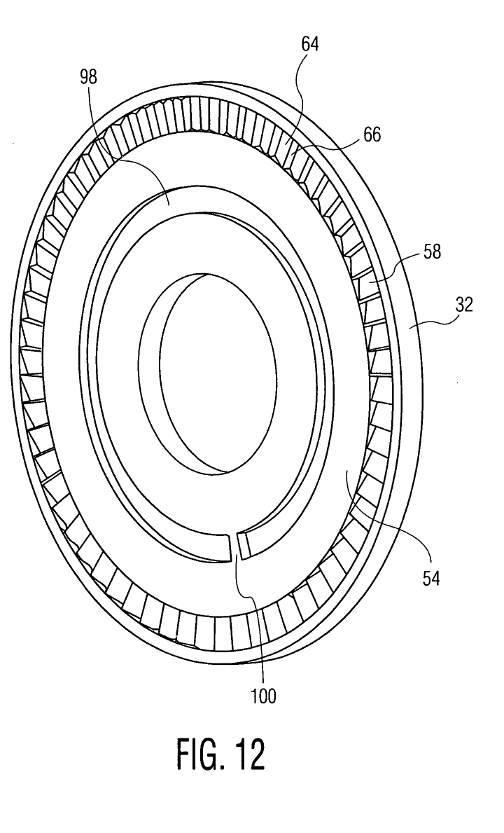

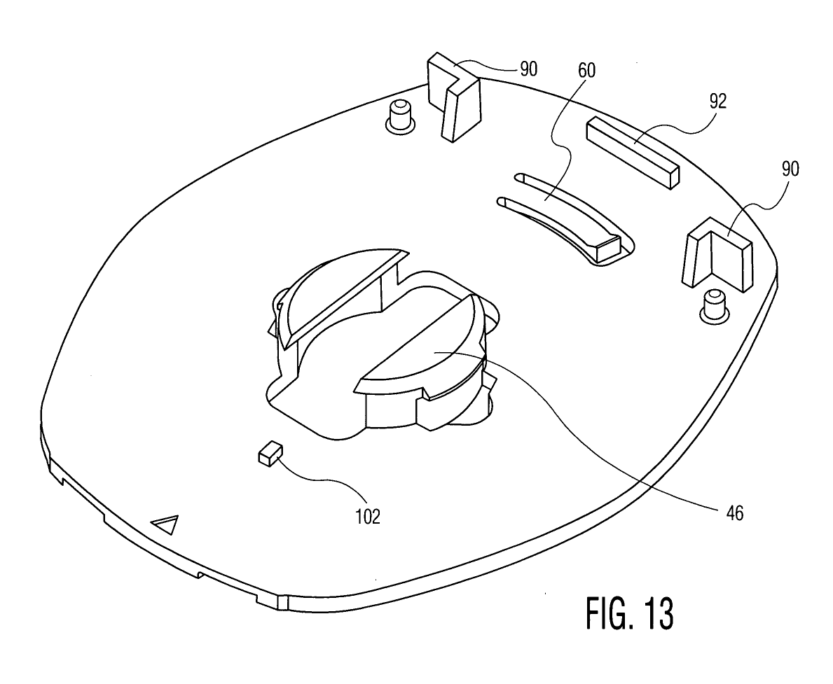

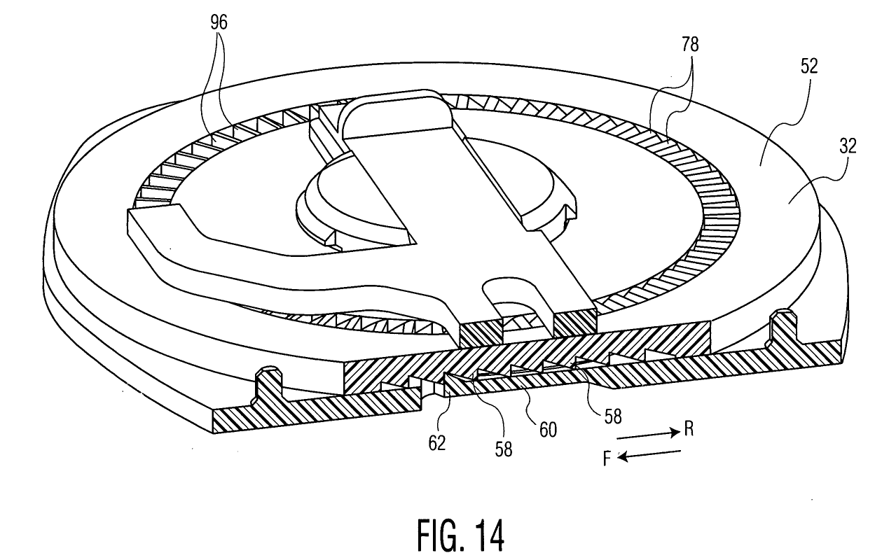

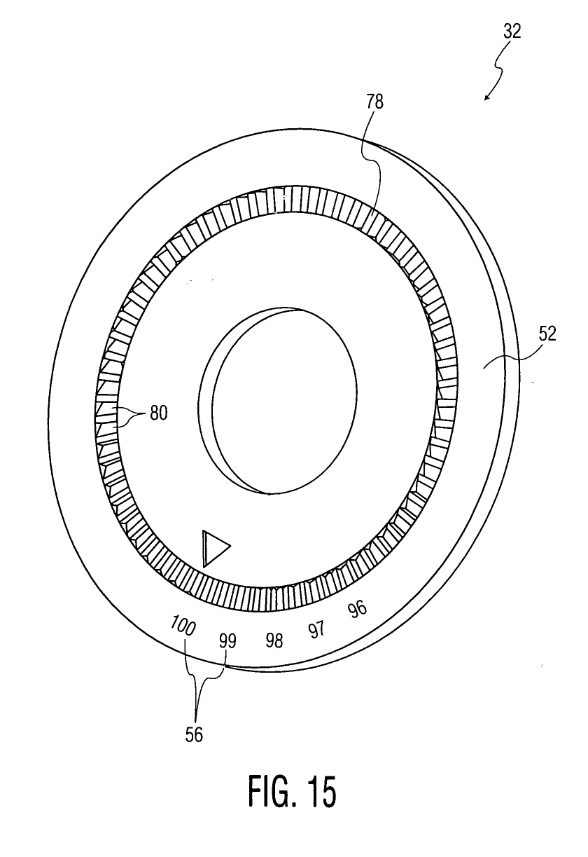

With reference to FIG. 11, the counter 32 is rotatably attached to the housing 14 in the same manner as in the first variation. As with the first configuration, it is preferred that a mechanism be provided to permit one-way rotation of, and to provide a releasable locking effect, to the counter 32. To this end, by way of non-limiting example, the ratchet teeth 58 may be provided on the lower face 54 of the counter 32, as shown in FIG. 12. In the manually adjustable configuration, the ratchet teeth 58 are oriented to point towards the inner opening 44 of the counter 32; in the second variation, the ratchet teeth 58 are oriented to point towards the lower base 36 of the housing 14. The indexer 60 extends from the housing 14 (FIG. 13), particularly from the lower base 36. As shown in FIG. 14, the indexer 60 is biased to have the end 62 nest between a pair of the ratchet teeth 62 to prevent rearward rotation (represented by the arrow R) yet allow forward rotation (represented by the arrow F) under proper and sufficient force of rotation in the same manner described above with respect to the first variation.

A series of driving ratchet teeth 78 are provided on the upper face 52 of the counter 32. The driving ratchet teeth 78 are shaped in the same manner as the ratchet teeth 58. Preferably, the driving ratchet teeth 78 have driving sloped surfaces 80 oriented to provide the same effect as the ratchet teeth 58. Specifically, as shown in FIG. 14, the driving sloped surfaces 80 are oriented to permit rotation in the direction represented by the arrow F but to prevent rotation in the direction represented by the arrow R. With this arrangement, the ratchet teeth 62 and the driving ratchet teeth 78 prevent rearward rotation yet permit forward rotation as described below.

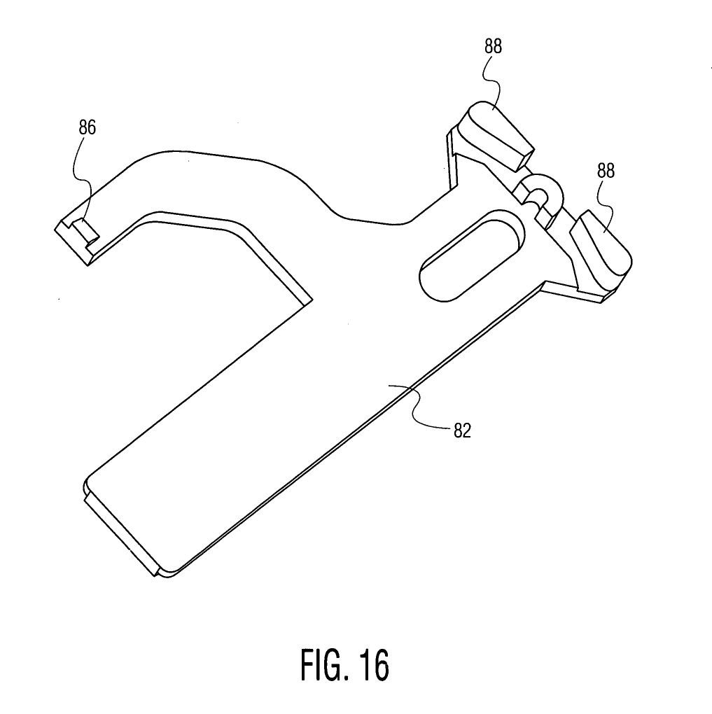

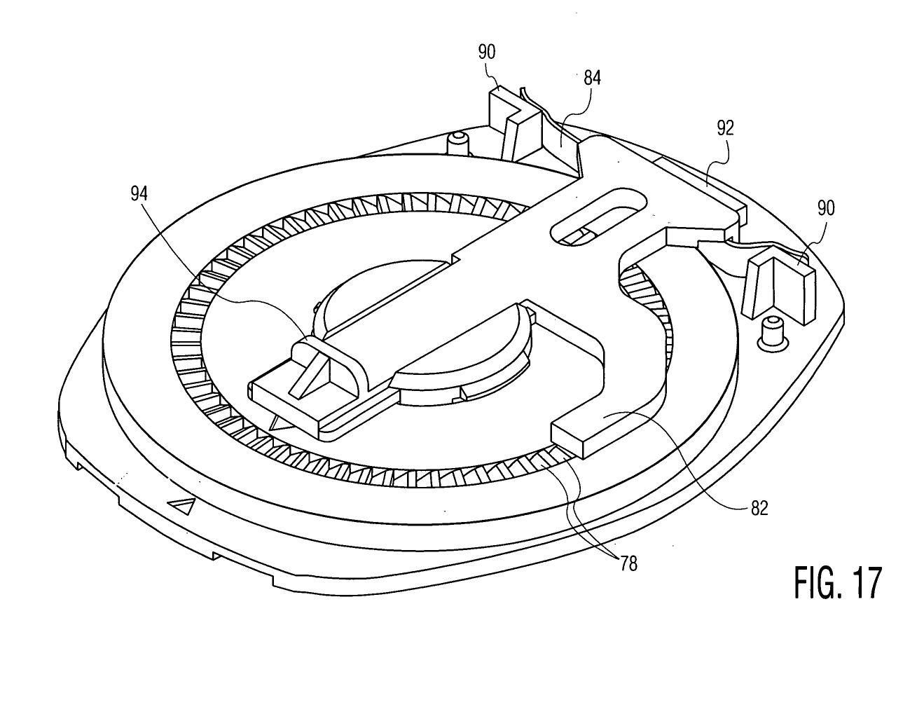

Any mechanism for providing force to cause automatic adjustment of the counter 32 may be used with the subject invention. With the second variation, a slider 82 and biasing means 84 are utilized. The slider 82 includes a biased driving detent 86 (FIG. 16) positioned and configured to nest between a pair of the driving ratchet teeth 78 (FIG. 17). The driving detent 86 is formed to coact with the driving ratchet teeth 78 in the same manner as the end 62 of the indexer 60 coacts with the ratchet teeth 58.

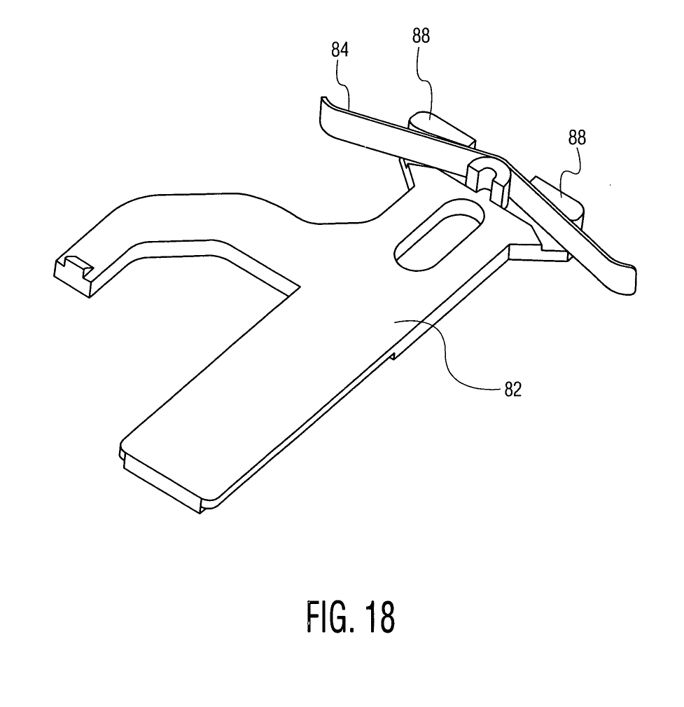

The biasing means 84 is fixed to the housing 14 in any known manner to urge the slider 82 to a rest position. The biasing means 84 may be a leaf spring, as shown in FIG. 18. Posts 88 may extend from the slider 82 against which the biasing means 84 may act. Spring restrictors 90 may extend from the housing 14 (FIG. 13) to hold the biasing means 84 in place. A stopper 92 may also extend from the housing 14 to limit the travel of the slider 82 under force of the biasing means 84. When assembled, as shown in FIG. 17, the biasing means 84 urges the slider 82 towards the stopper 92 to the rest position. It is preferred to have the slider 82 be urged to the rest position in a direction opposite the direction the dispenser 12 is inserted into the aperture 24 of the housing 14.

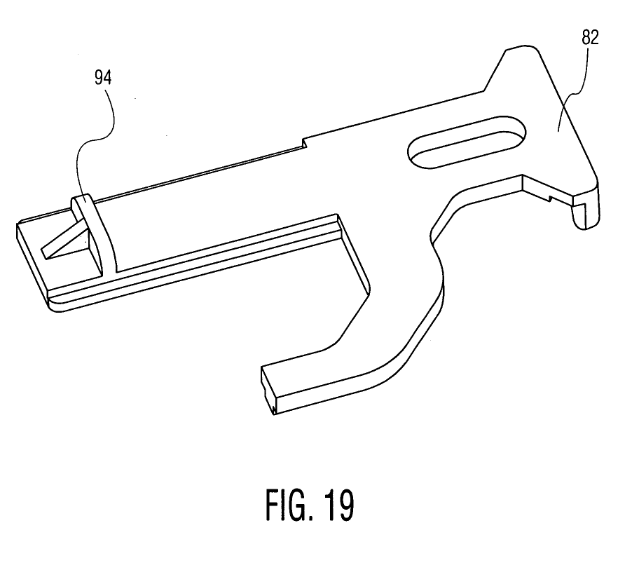

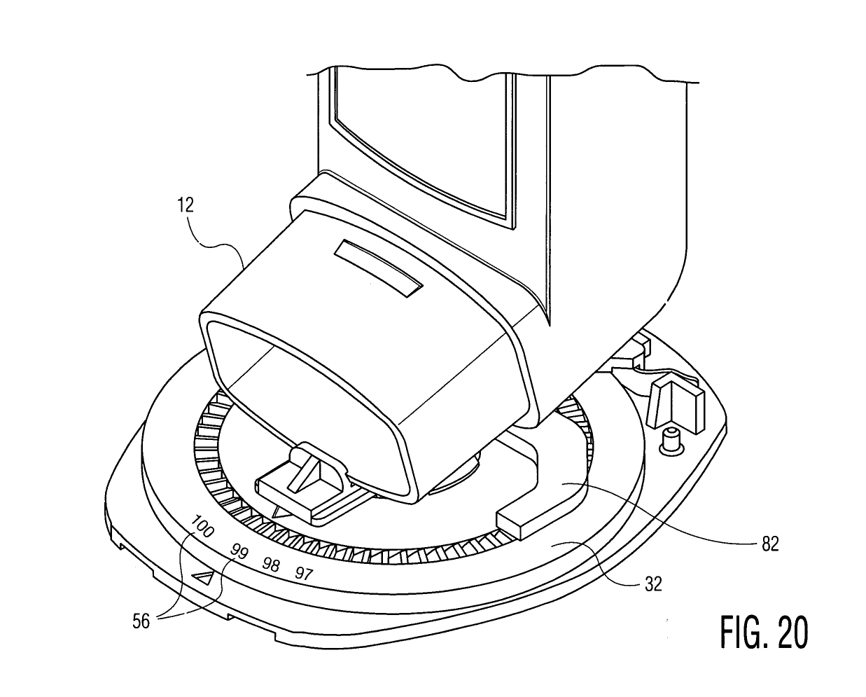

The slider 82 is provided with an upwardly extending engagement tab 94 (FIG. 19). The housing 14 is formed to have the engagement tab 94 engaged by the dispenser 12, as shown in FIG. 20, with the dispenser 12 being attached to the housing 14. This may be achieved by having the housing 14 open under the canopy 26. With the dispenser 12 being attached to the housing 14, the slider 82 is held by the dispenser 12, against the force of the biasing means 84, in a ready position (spaced from the rest position).

In use, the dispenser 12 is separated from the housing 14 for dose administration. With the dispenser 12 being removed from the engagement tab 94, the biasing means 84 urges the slider 82 to the rest position. Under force of this movement, the driving detent 86 ascends the driving sloped surface 80 of one of the driving ratchet teeth 78. The counter 32 is prevented from rotating during this motion by the interengagement of the indexer 60 and the ratchet teeth 58. Specifically, the ratchet teeth 58 are configured to prevent rotation of the counter 32 as the slider 30 moves to the rest position. With the slider 82 moving to the rest position, the counter 32 does not move. The slider 82 remains in the rest position, until the dispenser 12 is attached to the housing 14 after dose administration. Upon attachment, the dispenser 12 engages the engagement tab 94. As the dispenser 12 is driven to an attached position, the dispenser 12 pushes the engagement tab 94 from the rest position towards the ready position against the urging of the biasing means 84. Under force of this motion, the driving detent 86 pushes against a driving back surface 96 (FIG. 14) of one of the driving ratchet teeth 78. The driving detent 86 is formed to not ascend the back surface 96, and the interengagement of the driving detent 86 and the driving back surface 96 causes the counter 32 to rotate. Simultaneously, the indexer 60 is caused to traverse one of the ratchet teeth 58. Preferably, the motion of the slider 82 from the rest position to the ready position coincides with the indexer 60 traversing one of the ratchet teeth 58. With rotation of the counter 32, the indicia 56 are adjusted relative to the window 28 formed in the housing 14.

The lock 70 may be utilized to lock the dose counter 30 at the depletion of doses in the dispenser 12. Various lock arrangements may be utilized. With reference to FIG. 12, the counter 32, particularly the lower face 54, may be provided with a limiting groove 98. The limiting groove 98 terminates at a stop surface 100. A key 102 (FIG. 13) extends from the housing 14 shaped and positioned to extend into the limiting groove 98. In a start position of the dose counter 30 (i.e., a position coinciding with the beginning of the dose counting), the key 102 is located away from the stop surface 100. With operation of the dose counter 30, the counter 32 rotates with the key 102 approaching the stop surface 100. The key 102 eventually engages the stop surface 100 with further rotation being inhibited. At the same time, the configuration of the ratchet teeth 58 and the driving ratchet teeth 78 prevents rearward rotation. In this manner, rotation in either rotational direction is prohibited. As with the first configuration, the lock 70 need not be utilized so as to permit re-use of the housing 14. In addition, the lock 70 may be formed to be re-settable to permit re-use (e.g., permit relocation of the key 102 in the limiting groove 98 to the start position).

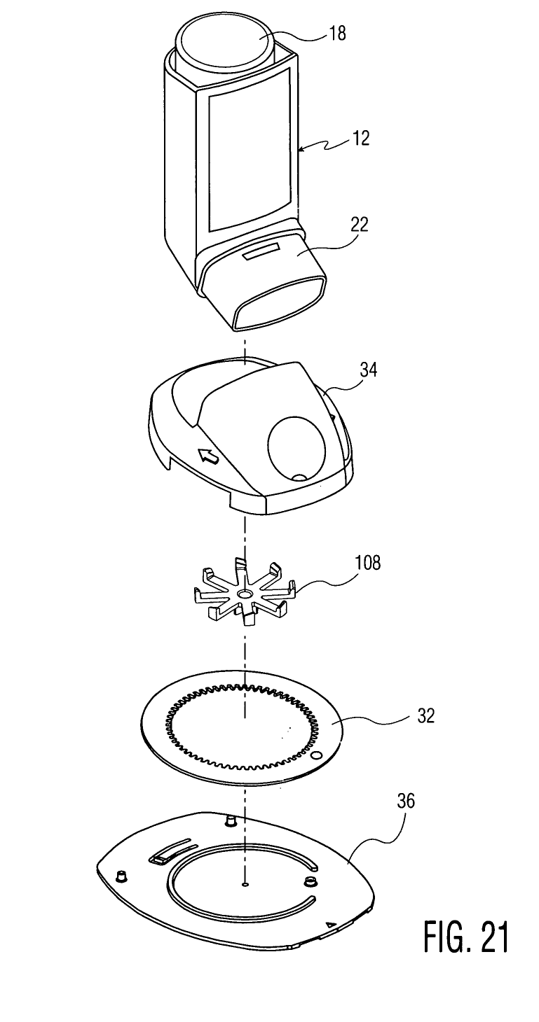

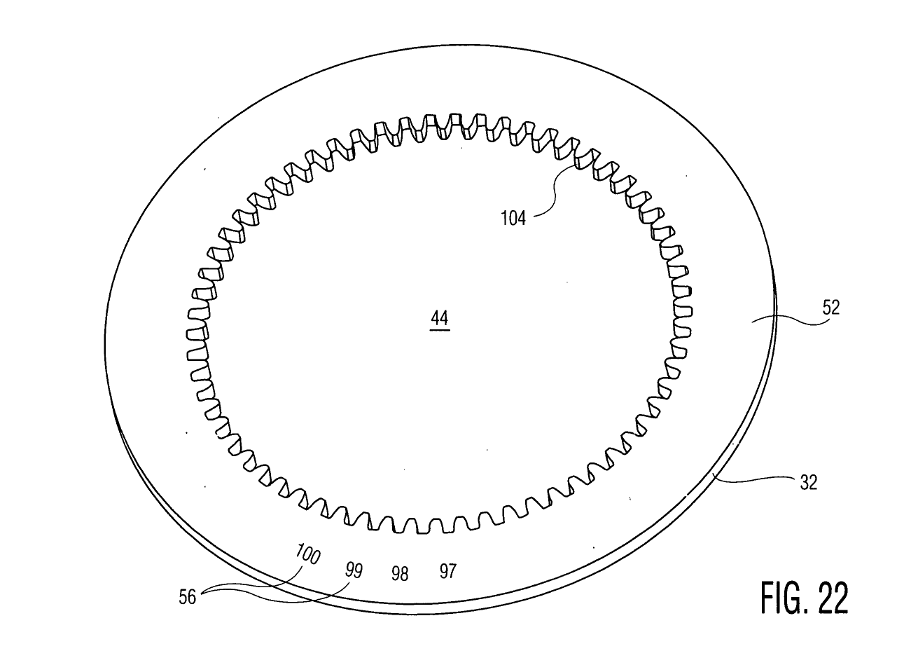

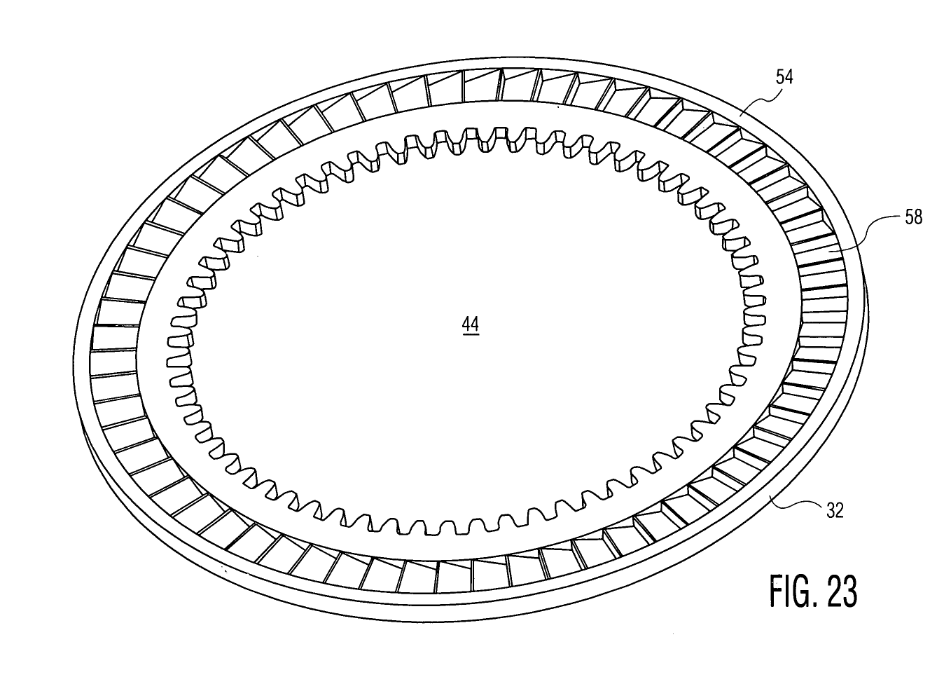

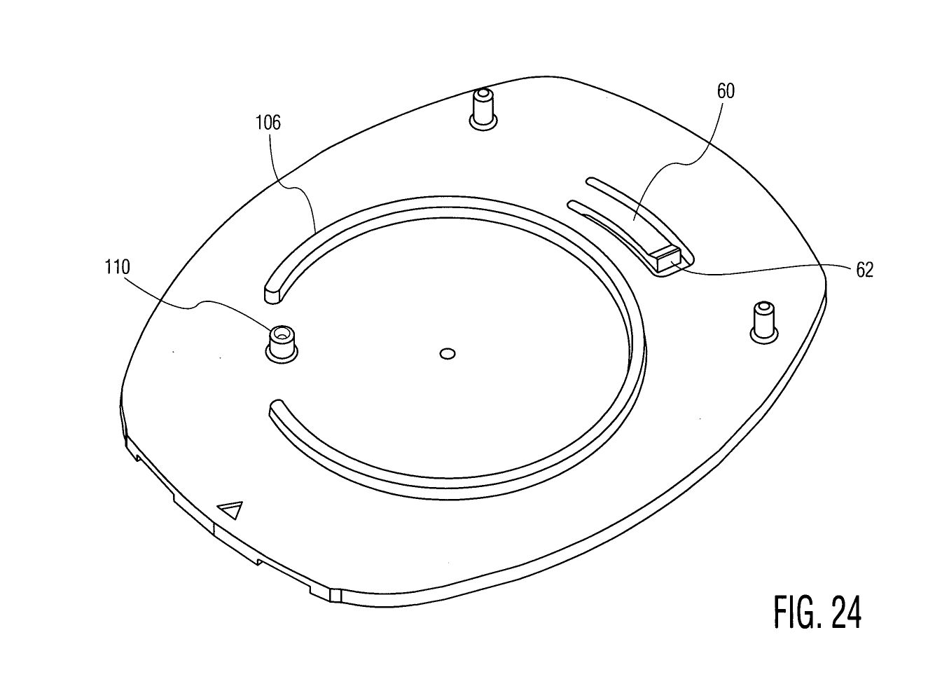

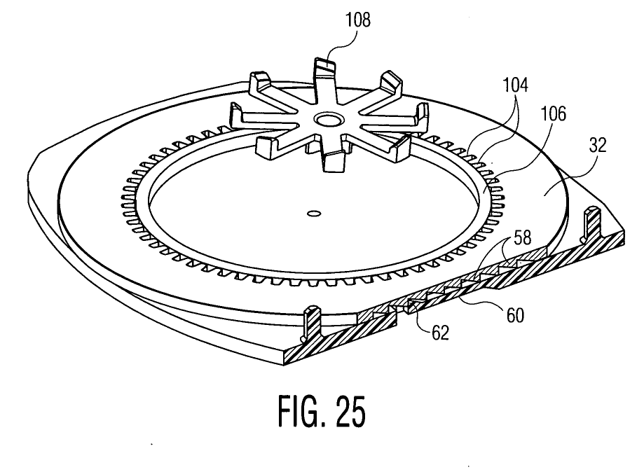

A third variation of the invention provides a different mechanism for automatically causing adjustment of the dose counter 30. With reference to FIGS. 21-23, in this variation, the inner opening 44 of the counter 32 is formed with gear teeth 104. Also, the lower face 54 of the counter 32 may be formed with the ratchet teeth 58 to provide a mechanism for limiting one way rotation and to provide a releasable locking effect, same as with the second variation. The indexer 60 may extend from the housing 14 (FIG. 24). As shown in FIG. 25, the ratchet teeth 58 and the indexer 60 are configured and operate in the same manner as in the second variation.

In the third variation, the counter 32 is preferably not latched to the housing 14. Rather, the counter 32 lies on the lower base 36. To minimize movement of the counter 32, a guide ring 106 may be defined in the housing 14, which may be continuous or discontinuous. The guide ring 106 may be sized to fit within the gear teeth 104, as shown in FIG. 25, with the counter 32 being rotatable about the guide ring 106.

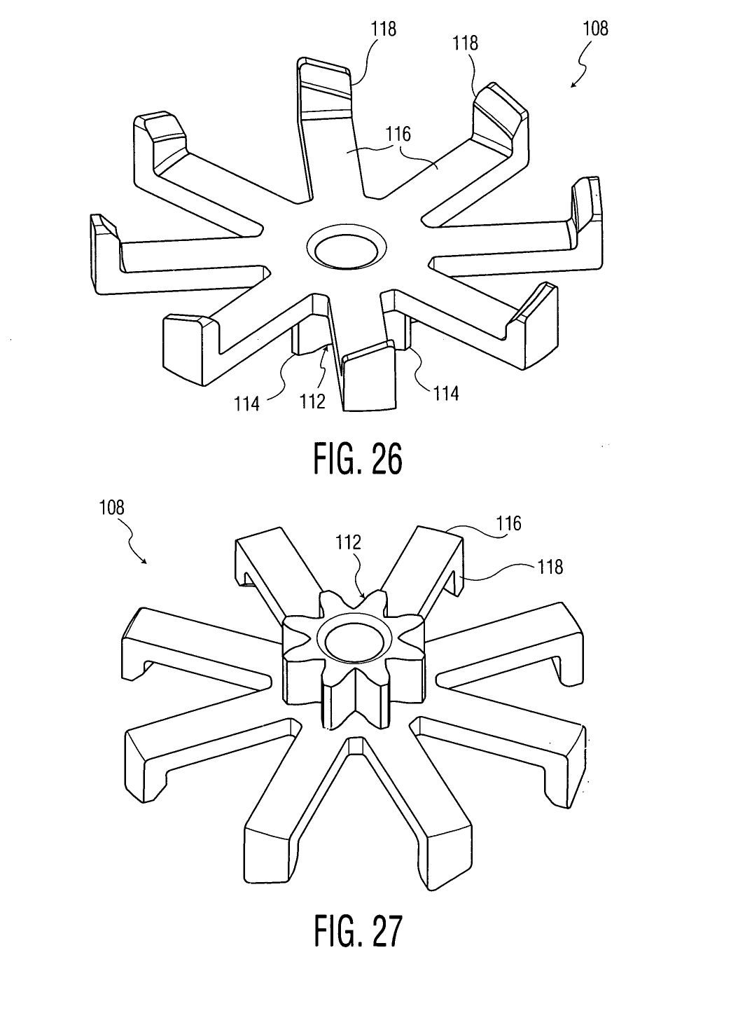

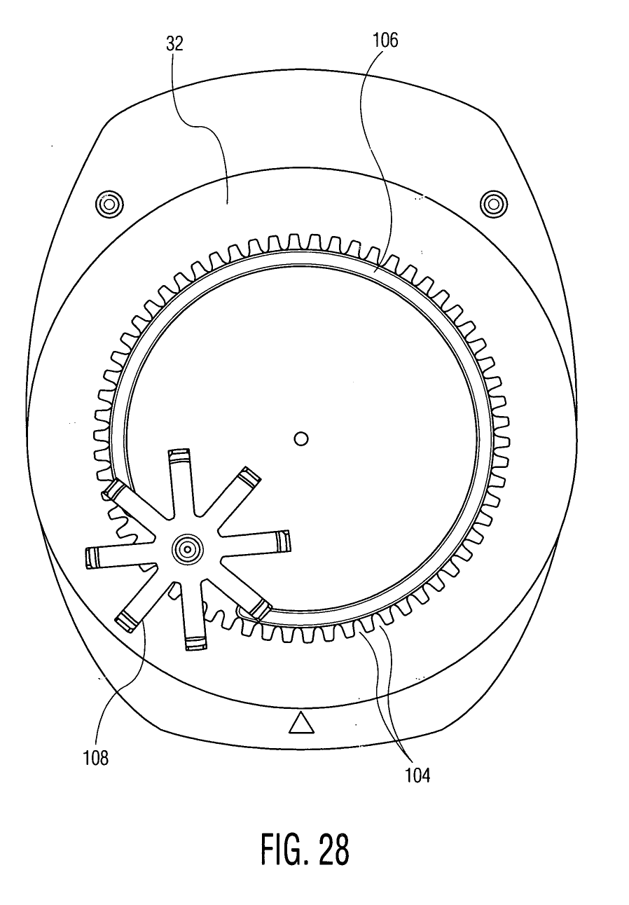

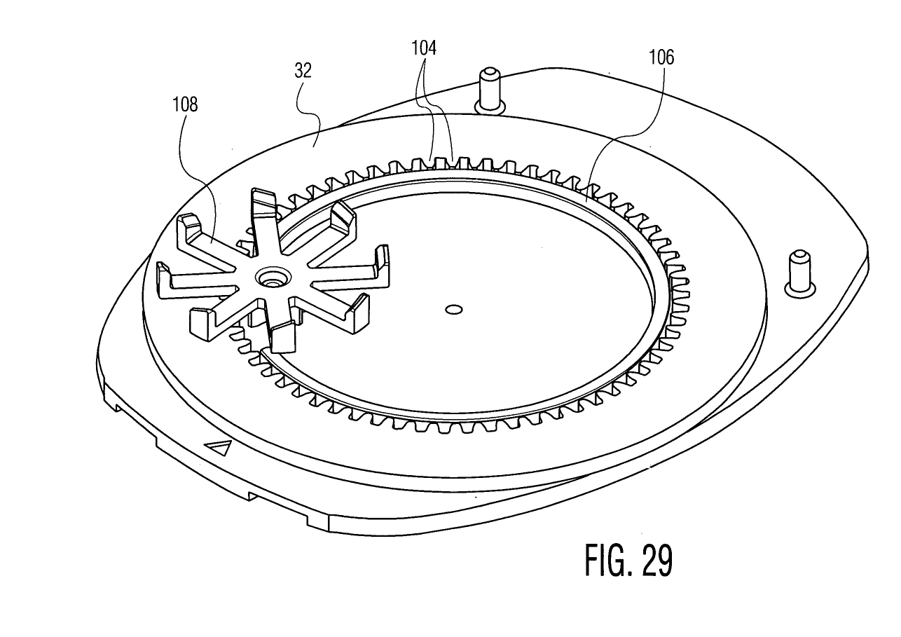

The third configuration includes a driving wheel 108 which is rotatably mounted to the housing 14 such as at shaft 110 (FIG. 24). The driving wheel 108 has a lower drive gear 112 having drive teeth 114 formed to mesh with the gear teeth 104 of the counter 32 (FIGS. 26-27). The driving wheel 108 is positioned in the housing 14 to have the drive teeth 114 meshingly engage the gear teeth 104 so that rotation of the driving wheel 108 results in rotation of the counter 32 (FIGS. 28-29). The guide ring 106 is preferably interrupted in proximity to the shaft 110 to permit proper location and operation of the driving wheel 108.

With reference to FIGS. 26 and 27, the driving wheel 108 is formed with a plurality of arms 116 extending from the center thereof. The arms 116 preferably terminate with upwardly extending segments 118. The segments 118 are spaced out about the periphery of the driving wheel 108.

The housing 14 and the dose counter 30 are assembled so that the dispenser 12 may engage the driving wheel 108, more particularly so that the dispenser 12 may engage one of the arms 116 upon being attached to, the housing 14. As with the second variation, the housing 12 may be formed open below the canopy 26 to provide access to the driving wheel 108.

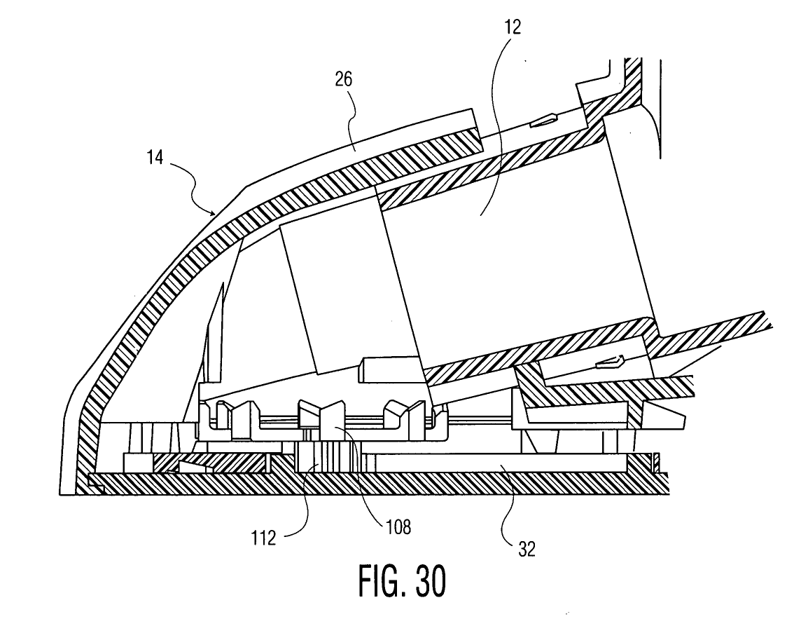

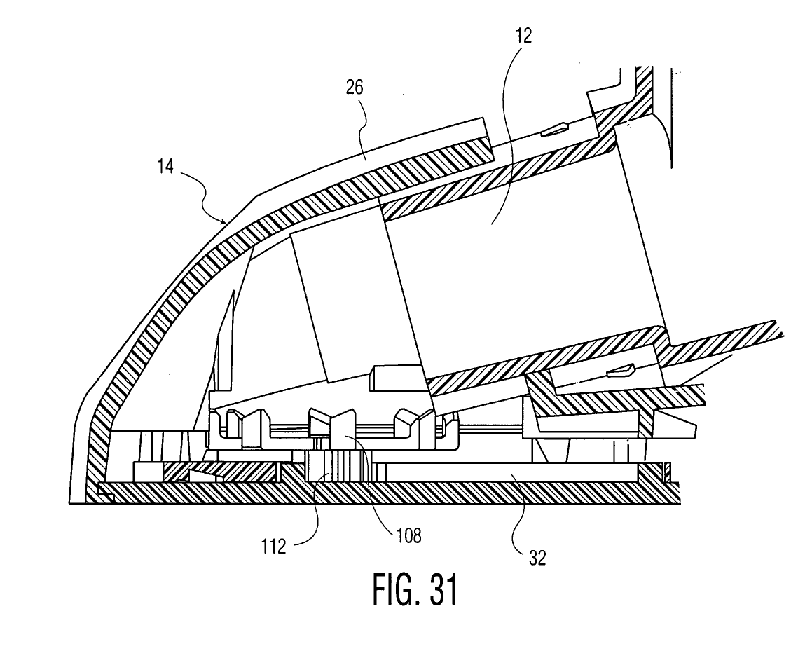

In use, the dispenser 12 is separated from the housing 14 to administer a drug dosage. With reference to FIGS. 30-32, upon attaching the dispenser 12 to the housing 14, the dispenser 12 engages one of the arms 116. With forward insertion of the dispenser 12 into the housing 14, the arm 116 of the driving wheel 108 is forced forwardly. At the same time, the drive gear 112 forces the counter 32 to rotate. The driving wheel 108, the housing 14 and the dispenser 12 are positioned and arranged to have the counter 32 only rotate one increment relative to the indicia 56 upon attaching the dispenser 12 to the housing 14. The ratchet teeth 58 and the indexer 60 are configured to limit rotation of the counter 32 to the one increment. Each administered dose can be counted in this manner. The lock 70 may be also provided as described above.

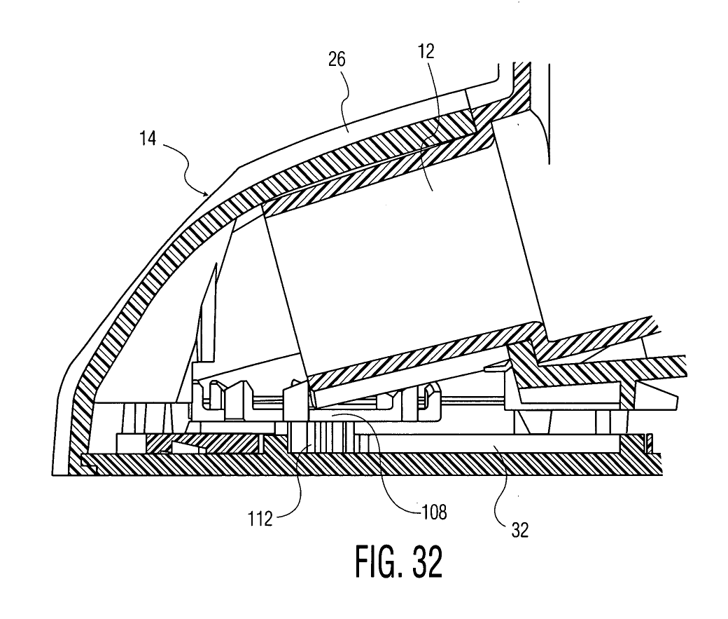

As will be readily appreciated by those skilled in the art, variations to the described configurations are possible. For example, the indicia 56 may be displayed by electronic means rather than by analog means. For example, an electronic display may be used which receives a signal with each incremental rotation of the counter 32. In addition, the dose counter 30 may be completely electronic, wherein the counter 32 is not utilized. By way of non-limiting example, a fourth variation is presented in FIGS. 32-37 where the dose counter 30 is completely electronic and is automatically adjusted.

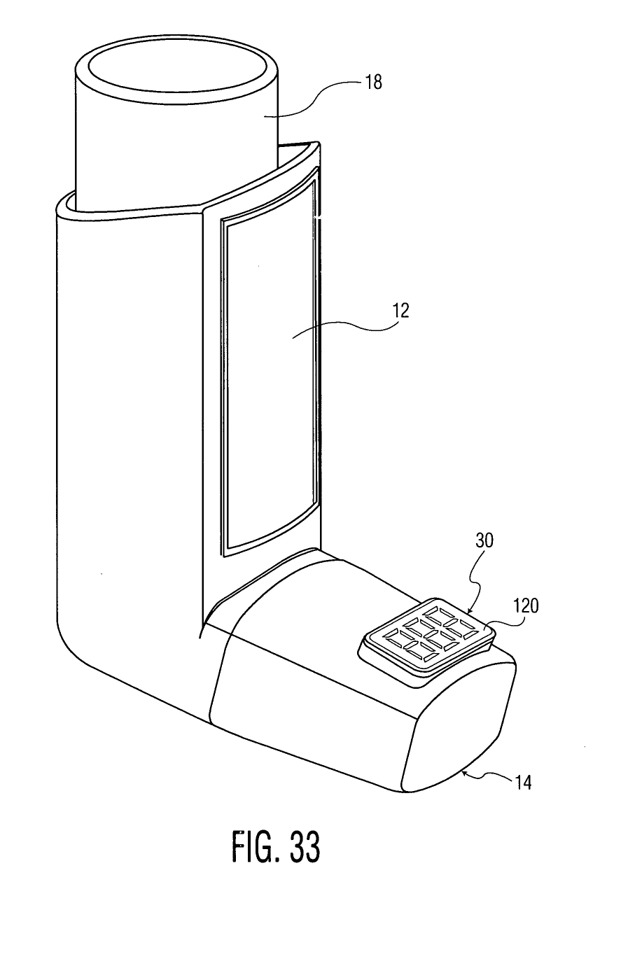

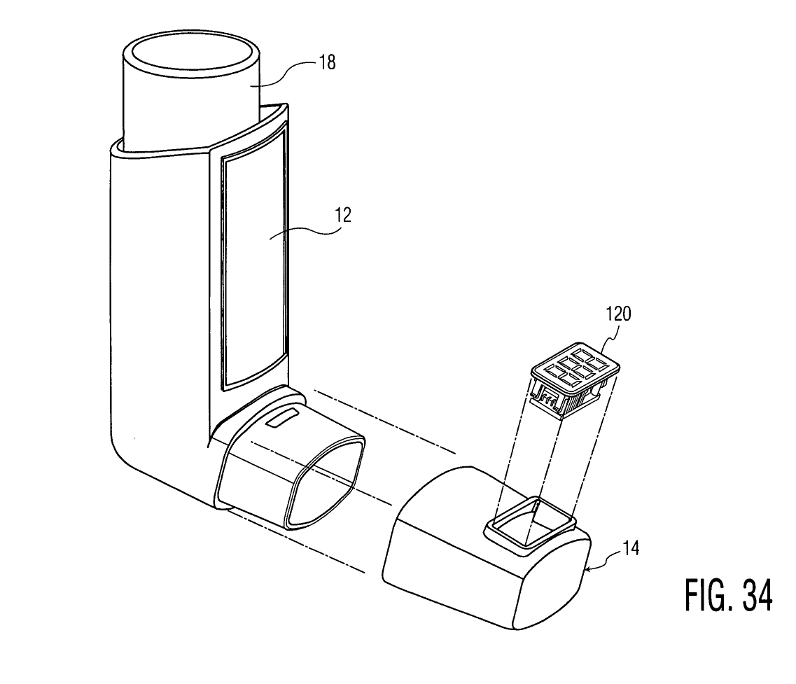

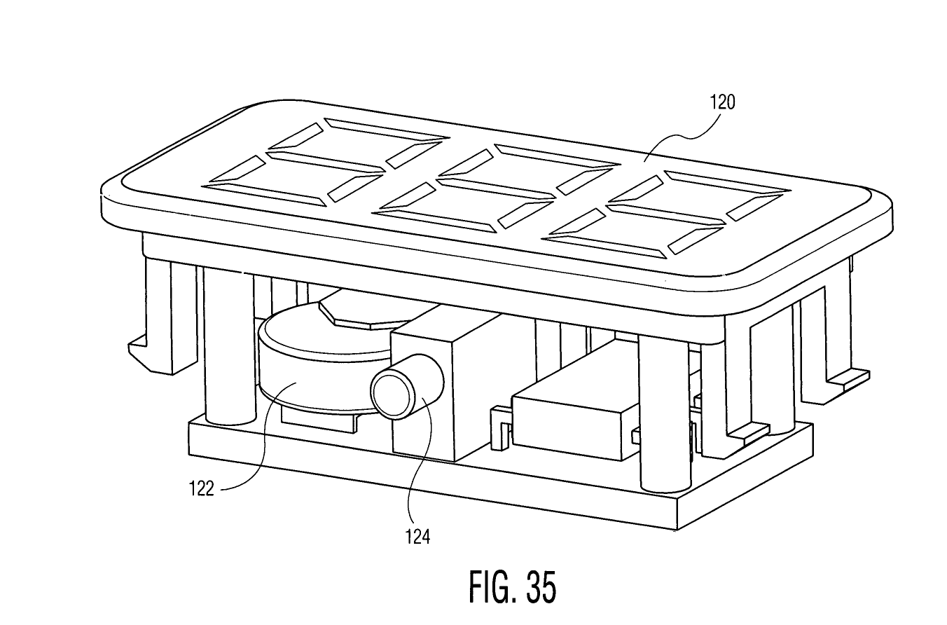

With reference to FIGS. 33-34, the dose counter 30 is operatively connected to the housing 14. The dose counter 30 includes an electronic display 120 mounted into a sidewall of the housing 14 for displaying the indicia 56. As shown in FIG. 35, the electronic display 120 is electrically connected to an electronic counter 122 and a plunger 124. The electronic counter 122 may be of any configuration which permits incremental counting or ordering of data. The values generated by the electronic counter 122 are displayable by the electronic display 120 as the indicia 56. The plunger 124 is slidable and preferably biased to extend out of the electronic counter 122.

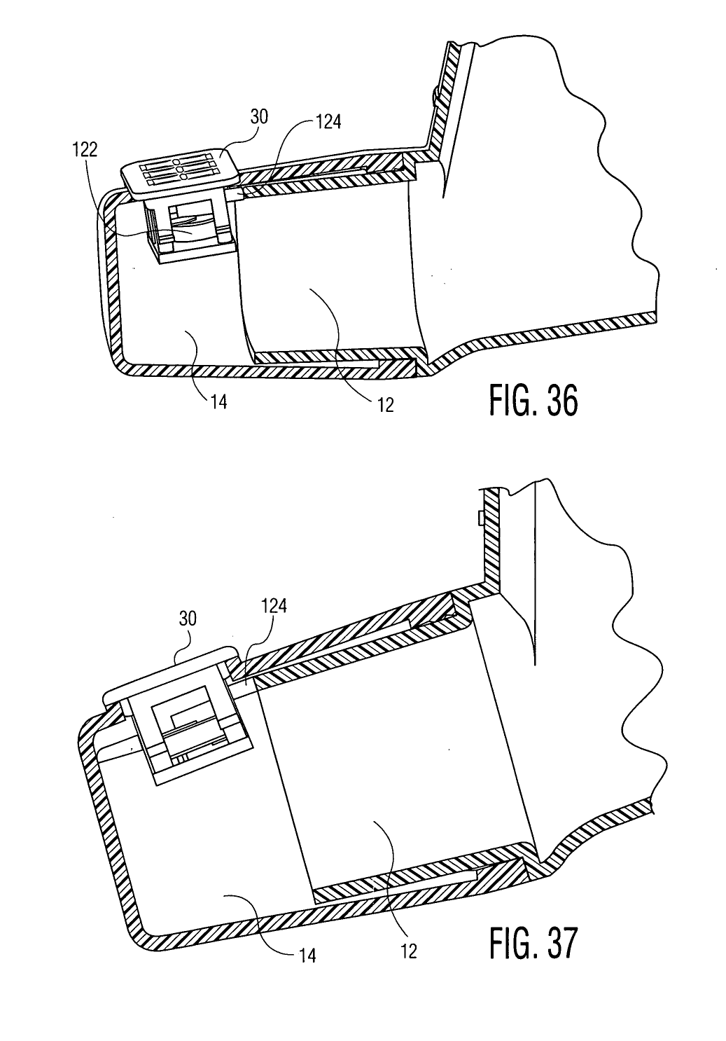

As shown by FIGS. 36-37, the dose counter 30 is positioned in the housing 14 to have the plunger 124 be engaged by the dispenser 12 with the dispenser 12 being attached to the housing 14. With the dispenser 12 attached to the housing 14, the plunger 124 is pushed into the electronic counter 122. With detachment of the dispenser 12 from the housing 14, the plunger 124 drives forward (away from the electronic counter 122) under force of the internal bias. The electronic counter 122 may be configured to count one increment (i.e., one dose administered) with the plunger 124 being driven out of the electronic counter 122 (i.e., count upon detachment) or to count one increment with the plunger 124 being driven into the electronic counter 122 (i.e., count upon attachment).

The plunger 124 can be replaced by a non-contact sensor which avoids the need for direct contact between the dispenser 12 and the plunger 124. By way of non-limiting examples, a magnetic sensor, an infrared sensor, a trippable light beam, etc. may be utilized to send a signal to the electronic counter 122.

The dose counter 30 can be provided with various features, such as a reset button to permit reuse; a timer to measure time intervals between doses; an audible or visual indicator (electronic beeper or light) to indicate time for dosing; an audible or visual indicator to indicate low dosage levels; etc. In addition, the dose counter 30 can be electronic and manually adjustable by providing a button or switch which allows a user to manually adjust the indicia 56 on the electronic counter 122. With this arrangement, the plunger 124 or other sensor is not needed.

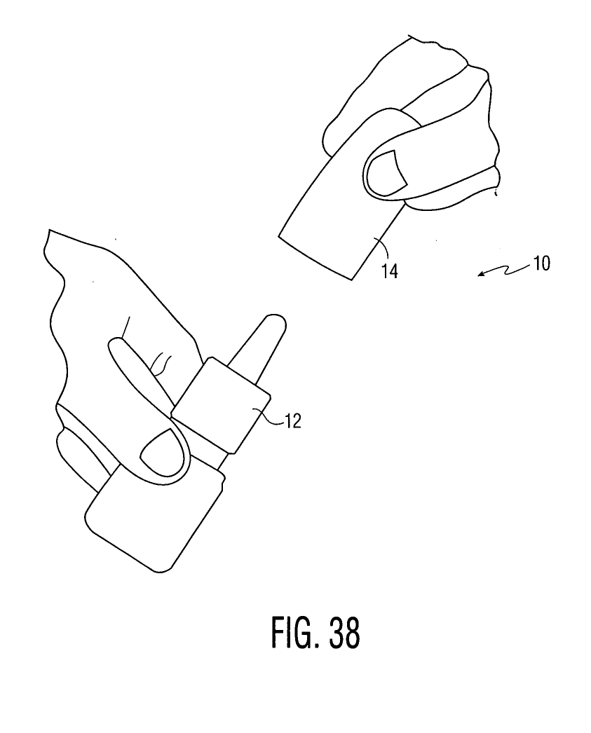

FIG. 38 depicts the dispenser 12 as a nasal inhaler.

It may be desired to provide the kit 10 with means to associate the dispenser 12 with the housing 14. Disassociation of the two may be a concern where a user utilizes a plurality of the dispensers 12. By matching the housing 14 inadvertently with a non-corresponding dispenser 12, a false indication of dosage amount may result. To limit disassociation, and with reference to FIG. 39, a flexible tether 126 or other linking member may be fixed to the dispenser 42 and the housing 14. The tether 126 preferably has sufficient length to promote unobstructed use of the dispenser 12. The housing 14 may be directly secured to the dispenser 12; for example, the housing 14 may be pivotally attached to selectively permit access to the dispense opening 20. Alternatively, the dispenser 12 and the housing 14 may be color matched or provided with matching insignia to indicate a pair. Optionally, the dispenser 12 and the housing 14 may be formed with a specific mating configuration (e.g., the aperture 24 and the mouthpiece 22 are specifically shape mated); different shape mating configurations for different combinations of the dispenser 12 and the housing 14 (e.g., different configurations for different types of drugs) may be utilized over a plurality of the kits 10 to minimize the possibility of disassociation.

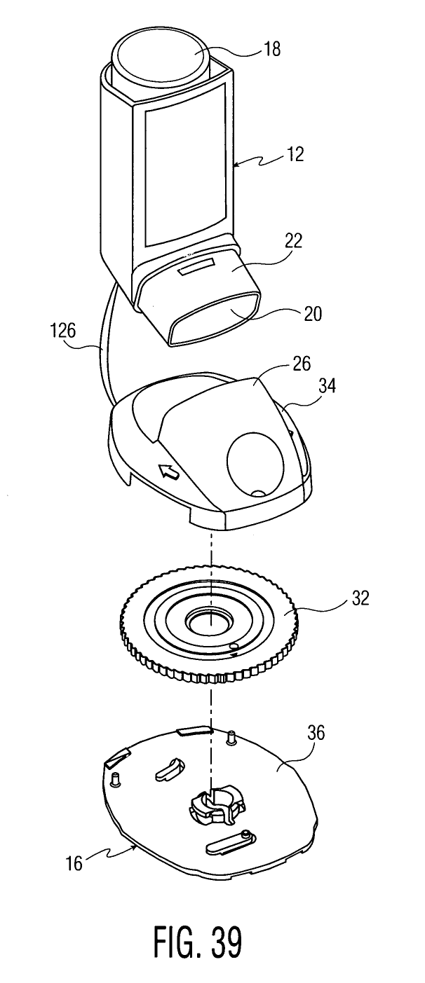

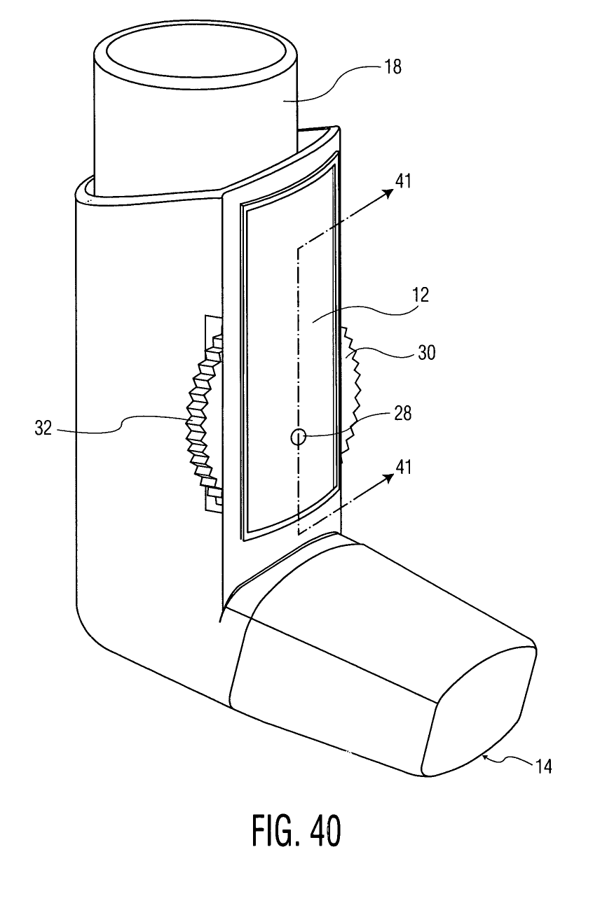

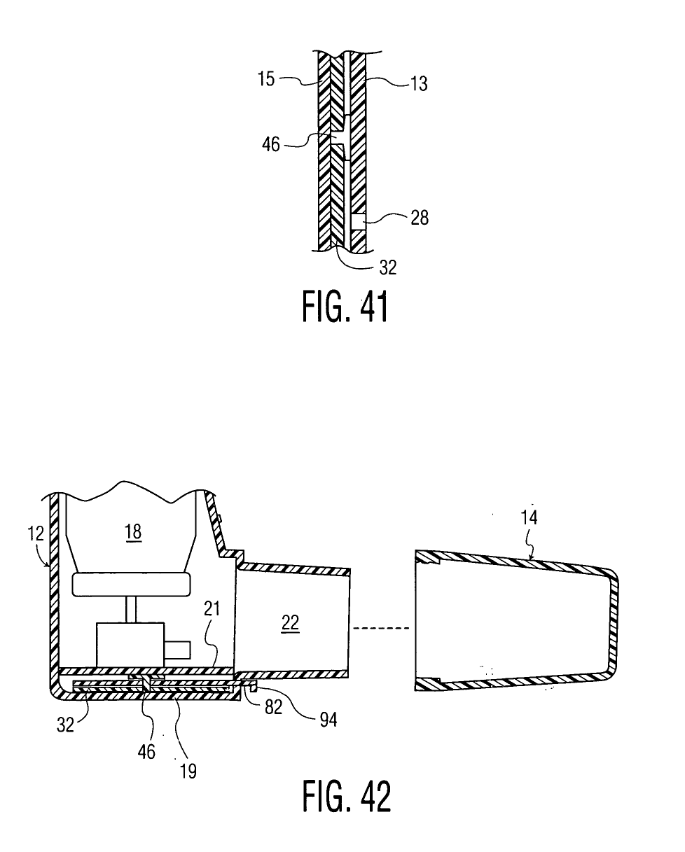

As will be appreciated by those skilled in the art, the dose counter 30 described herein may be operatively connected to the dispenser 12, rather than the housing 14. The dose counter 30 may be detachably attached to, or formed integrated with, the dispenser 12. For example, and with reference to FIGS. 40 and 41, with respect to the first variation, the dose counter 30 may be mounted to the dispenser 12 to be manually adjustable. In particular, the counter 32 may be rotatably mounted, e.g. by the latches 46, to an outer wall 13 and/or an inner wall 15 of the dispenser 12.

With respect to the second through fourth configurations, the dose counter 30 may be mounted to the dispenser 12 and rely on interengagement with the housing 14 in the same manner as the embodiments described above rely on interengagement with the dispenser 12. The housing 14 will be provided regardless of how the dose counter 30 is formed or secured.

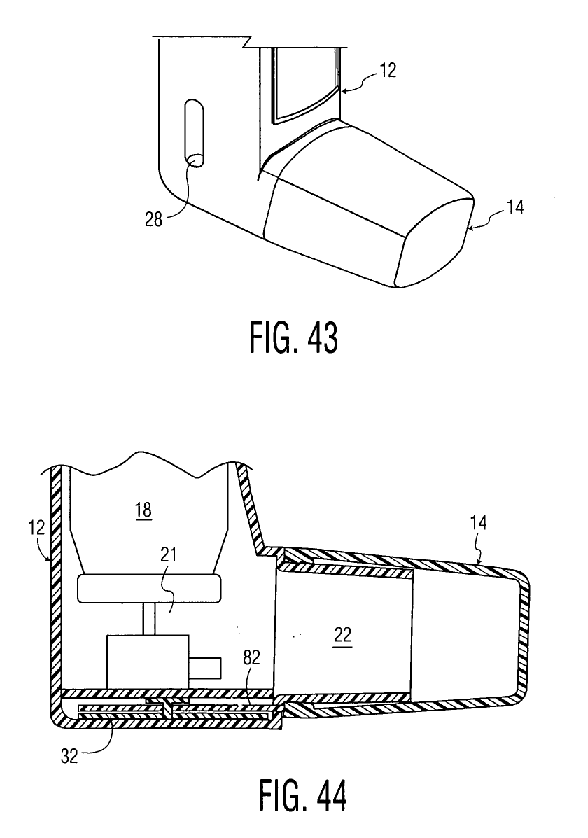

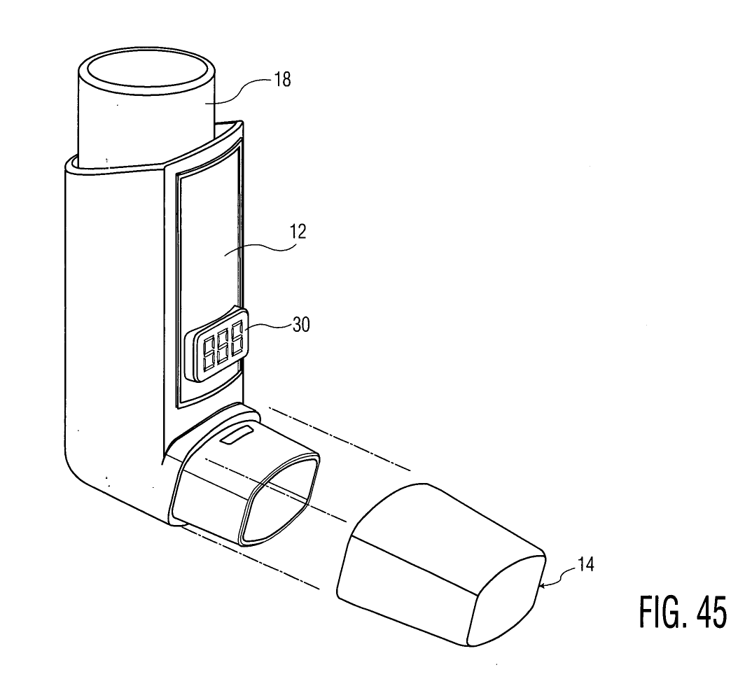

With specific reference to FIGS. 42-44, the second variation may be provided in between base 19 and raised floor 21 of the dispenser 12. The slider 82 extends from the dispenser 12 with the engagement tab 94 being exposed. With the housing 14 being mounted onto the dispenser 12, particularly about the mouthpiece 22, the housing 14 engages the engagement tab 94 and causes rearward displacement thereof, along with the slider 82. Displacement of the slider 82 causes advancement of the counter 32 in the same manner as described above. As shown in FIG. 43, the indicia 56, which face upwardly towards the reservoir 18 in this arrangement, may be viewed through the window 28 formed in a side of the dispenser 12. The third variation may likewise be housed between the base 19 and the raised floor 21 and operate as described above.

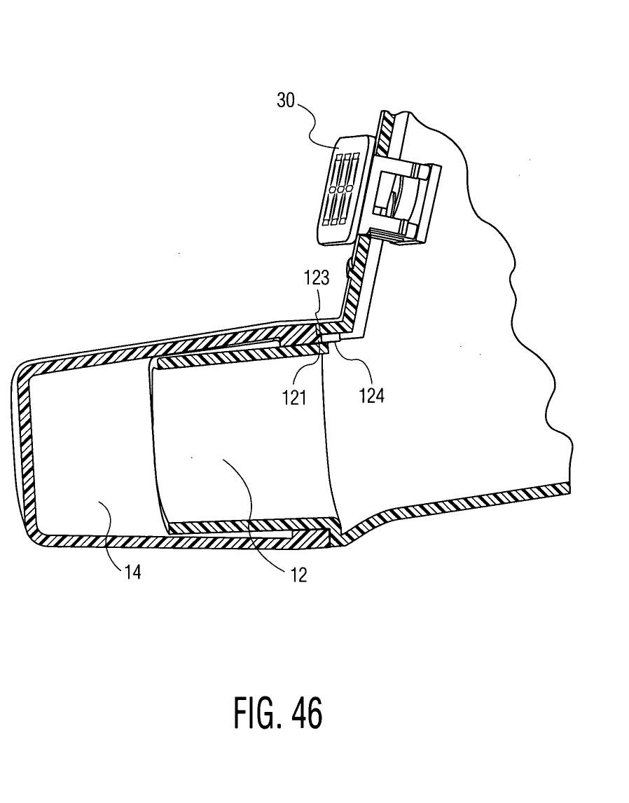

FIGS. 45-46 depict the fourth variation with the dose counter 30 being mounted to the dispenser 12. The plunger 124 is located to be engaged by the housing 14. For example, a protrusion 121 may be provided on the housing 14 formed to register with an opening 123 formed in the dispenser 12. The plunger 124 is positioned to be engaged by the protrusion 121, with the protrusion 121 extending through the opening 123. Other portions of the housing 14 may be used to engage the plunger 124. The plunger 124 is electronically coupled to the dose counter 30 to provide signals for actuation thereof for counting doses as described above.

What is claimed is:

1. An apparatus for counting doses of a dispenser, said apparatus comprising:
a housing for releasably attaching to the dispenser; and,
a manually adjustable dose counter operatively connectable to the dispenser,
said dose counter displaying indicia relating to doses of the dispenser.

2. An apparatus as in claim 1 further comprising means for releasably locking said dose counter to selectively prevent adjustment thereof.

3. An apparatus as in claim 1, wherein the dispenser is an oral inhaler or nasal inhaler.

4. An apparatus as in claim 1, wherein the dispenser includes a dispense opening through which the doses are administered, said housing configured to cover at least a portion of the dispense opening when attached to the dispenser.

5. An apparatus as in claim 1, wherein said indicia are located on said dose counter, said indicia being selectively viewable through said housing.

6. An apparatus as in claim 1, wherein said dose counter includes an electronic display, said indicia being displayed electronically on said electronic display.

7. An apparatus as in claim 1 further comprising means to permit unidirectional adjustment of said dose counter.

8. An apparatus for counting doses of a dispenser, said apparatus comprising:
a housing for releasably attaching to the dispenser;
an adjustable dose counter operatively connectable to the dispenser, said dose counter displaying indicia relating to doses of the dispenser; and,
means for automatically causing adjustment of said dose counter upon said housing being attached to, or detached from, the dispenser.

9. An apparatus as in claim 8 further comprising means for releasably locking said dose counter to selectively prevent adjustment thereof.

10. An apparatus as in claim 8, wherein the dispenser is an oral inhaler or nasal inhaler.

11. An apparatus as in claim 8, wherein the dispenser includes a dispense opening through which the doses are administered, said housing configured to cover at least a portion of the dispense opening when attached to the dispenser.

12. An apparatus as in claim 8, wherein said indicia are located on said dose counter, said indicia being selectively viewable through said housing.

13. An apparatus as in claim 8, wherein said dose counter includes an electronic display, said indicia being displayed electronically on said electronic display.

14. An apparatus as in claim 8 further comprising means to permit unidirectional adjustment of said dose counter.

15. An apparatus for counting doses of a dispenser, the dispenser having a dispense opening through which the doses are administered, said apparatus comprising:
a housing for releasably attaching to the dispenser, said housing configured to cover at least a portion of the dispense opening when attached to the dispenser; and
a dose counter operatively connectable to the dispenser, said dose counter displaying indicia relating to doses of the dispenser.

16. An apparatus as in claim 15, wherein the dispenser is an oral inhaler or a nasalinhaler.

17. An apparatus as in claim 16, wherein, with the dispenser being an inhaler, the dispenser includes a mouthpiece, the dispense opening being formed in the mouthpiece.

18. A kit comprising:
a drug reservoir;
a dispenser for dispensing doses from said drug reservoir; and
an apparatus for counting doses of said dispenser, said apparatus including:
a housing for releasably attaching to the dispenser; and,
a manually adjustable dose counter operatively connected to the dispenser, said dose counter displaying indicia relating to doses of the dispenser.

19. A kit comprising:
a drug reservoir;
a dispenser for dispensing doses from said drug reservoir; and
an apparatus for counting doses of said dispenser, said apparatus including:
a housing for releasably attaching to the dispenser;
an adjustable dose counter operatively connected to the dispenser, said dose counter displaying indicia relating to doses of the dispenser; and,
means for automatically causing adjustment of said dose counter upon said housing being attached to, or detached from, the dispenser.

20. A kit comprising:
a drug reservoir;
a dispenser for dispensing doses from said drug reservoir, said dispenser having a dispense opening through which the doses are administered; and
an apparatus for counting doses of said dispenser, said apparatus including:
a housing for releasably attaching to the dispenser, said housing configured to cover at least a portion of the dispense opening when attached to the dispenser; and
a dose counter operatively connected to the dispenser, said dose counter displaying indicia relating to doses of the dispenser.

21. A kit as in any of claims 18-20, wherein said drug cartridge includes a drug selected from the group consisting of an anticholinergic, a corticosteroid, a long acting beta agonist, short acting beta agonist, a phosphodiesterase IV inhibitor and combinations of two or more thereof;

22. A kit as in claim 21, wherein said anticholinergic is selected from the group consisting of (R)-3-[2-hydroxy-2,2-(dithien-2-yl)acetoxy]-1-1[2-(phenyl)ethyl]-1-azoniabicyclo[2.2.2]octane, Glycopyrrolate, Ipratropium Bromide, Oxitropium Bromide, Atropine Methyl Nitrate, Atropine Sulfate, Ipratropium, Belladonna Extract, Scopolamine, Scopolamine Methobromide, Methscopolamine, Homatropine Methobromide, Hyoscyamine, Isopriopramide, Orphenadrine, Benzalkonium Chloride, Tiotropium Bromide, GSK202405, an individual isomer of any of the above or a pharmaceutically acceptable salt or hydrate of any of the above, or a combination of two or more of the above.

23. A kit as in claim 21, wherein said corticosteroid is selected from the group consisting of Mometasone Furoate; Beclomethasone Dipropionate; Budesonide; Fluticasone; Dexamethasone; Flunisolide; Triamcinolone; (22R)-6.alpha., 9.alpha.-difluoro-11.beta., 21-dihydroxy-16.alpha., 17.alpha.-propylmethylenedioxy-4-pregnen-3,20-dione, Tipredane, GSK685698, GSK799943 or a pharmaceutically acceptable salt or hydrate of any of the above, or a combination of two or more of the above.

24. A kit as in claim 21, wherein said long acting beta agonist is selected from the group consisting of carmoterol, indacaterol, TA-2005, salmeterol, formoterol, or a pharmaceutically acceptable salt or hydrate of any of the above, or a combination of two or more of the above.

25. A kit as in claim 21, wherein said short acting beta agonist is selected from the group consisting of albuterol, terbutaline sulfate, bitolterol mesylate, levalbuterol, metaproterenol sulfate, pirbuterol acetate or a pharmaceutically acceptable salt or hydrate of any of the above, or a combination of two or more of the above.

26. A kit as in claim 21, wherein said phosphodiesterase IV inhibitor is selected from the group consisting of Cilomilast, Roflumilast, Tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above.

27. An apparatus or kit as in any of the preceding claims, wherein said indicia includes a series of adjacent numbers, said numbers serially descending by one in said series.

28. An apparatus or kit as in any of the preceding claims, wherein said indicia includes at least one colored field or background.
